SEQUENCE LISTING (19 sequences)

SEQ ID NO: 1 - DNA, 840 bp, Bacillus subtilis
atgacgcaattcgataaacaatacaattcaattataaaggatattatcaataatggaatctcagacgaagagtttgatgtaagaaccaagtgggactcagatggaacaccggcacatactctaagtgtaatcagtaagcaaatgagattcgacaactcagaggttccgattttaacgacaaaaaaggttgcctggaaaacagccattaaagagttgctctggatttggcagctgaaatctaatgatgttaatgatttaaacatgatgggcgtccatatttgggatcagtggaaacaagaagacggaaccatcggacatgcatatggatttcagctggggaagaaaaacagaagtctaaatggagaaaaagtggatcaggtagactatcttcttcatcaattgaagaacaatccatcttcacgcagacacattacaatgctgtggaatcctgatgaattagacgcaatggccttaacgccatgtgtatacgagacacaatggtacgttaaacatgggaaactccaccttgaggtaagagcacggagcaatgatatggcattgggaaatccattcaatgtattccagtataatgtgttgcagcgcatgattgctcaagtgactggttatgagcttggtgaatatatctttaacattggggattgccatgtgtacacacgtcatatagacaatttgaaaattcaaatggaaagagaacagtttgaagcacctgaactatggatcaatcctgaagtgaaagatttttatgactttaccattgatgatttcaagttaatcaactataaacatggggacaagcttttatttgaggtagcggtttaa

SEQ ID NO: 2 - DNA, 795 bp, Bacillus subtilis
atgaaacagtataaggatttctgcagacatgttttagagcatggtgagaaaaagggagaccggactgggaccggaacaatcagcactttcggatatcaaatgagatttaatttacgggaaggctttccgatgctcaccactaaaaaactccactttaaatcaattgcgcatgaactgctgtggttcttaaaaggagatacgaatgtacgctatctgcaggaaaacggagtgcgaatctggaatgagtgggctgatgaaaacggtgaacttggacctgtatatggctcccaatggcgttcttggcggggagctgatggagaaaccattgatcaaatttcccgtcttattgaagatattaaaacaaatccgaactccagacgcttaatcgtcagcgcctggaatgttggtgaaattgataaaatggcgttgccgccgtgccattgcctgttccaattctatgtgtctgacggcaagctgtcctgtcagctgtatcagcgctctgccgatgttttcttaggtgtgccgtttaatattgcatcttatgccctcctaaccatgatcattgctcatgtgactgggcttgaaccgggcgagttcatccatacgtttggtgatgttcatatttaccaaaatcatattgaacaagtcaatttgcagctggaaagagatgttagaccgcttccgcagcttcgtttcgccagaaaggttgattctatttttaactttgcatttgaggactttatcatcgaggattatgatccgcatcctcatataaaaggggcggtcagcgtatga

SEQ ID NO: 3 - DNA, 900 bp, Artificial Sequence
  left arm of thyA
cagatcatataaggaatgaaccgctgccaaatatcataaaaaagttgttaatgatcaaatgaatgaaattagagagaatttattttaaagaaagcccaattgcacatggacaaatgacaattgatattgaggtaaaatacaatgcattaaatcagaaaaagctattggaggatttaatgtgtttagacaatttccaatttggtatacacaaacacctgactatttgaatttttatgtaccgcaatatcaaaccatttcgtataatcctcaacaatgttatcaacggtgtatgtaccaaactggcggtaactatgagctatgtgacagactatgttatggagaaatacaggtgtaaaagagggggattaactcctctttaaacacacagtgagtggaataagatcctcactttatctgcaagtgcttagtatttgcgataatattgcattcgtaataaattatgcttagcaactgaaaatgaaagaaggatatgaatagtcatgacgcaattcgataaacaatacaattcaattataaaggatattatcaataatggaatctcagacgaagagtttgatgtaagaaccaagtgggactcagatggaacaccggcacatactctaagtgtaatcagtaagcaaatgagattcgacaactcagaggttccgattttaacgacaaaaaaggttgcctggaaaacagccattaaagagttgctctggatttggcagctgaaatctaatgatgttaatgatttaaacatgatgggcgtccatatttgggatcagtggaaacaagaagacggaaccatcggacatgcatatggatttcagctggggaagaaaaacagaagtctaaatggagaaaaagtggatcaggtagactatcttcttcatcaat

SEQ ID NO: 4 - DNA, 950 bp, Artificial Sequence
  right arm of thyA
tgaagaacaatccatcttcacgcagacacattacaatgctgtggaatcctgatgaattagacgcaatggccttaacgccatgtgtatacgagacacaatggtacgttaaacatgggaaactccaccttgaggtaagagcacggagcaatgatatggcattgggaaatccattcaatgtattccagtataatgtgttgcagcgcatgattgctcaagtgactggttatgagcttggtgaatatatctttaacattggggattgccatgtgtacacacgtcatatagacaatttgaaaattcaaatggaaagagaacagtttgaagcacctgaactatggatcaatcctgaagtgaaagatttttatgactttaccattgatgatttcaagttaatcaactataaacatggggacaagcttttatttgaggtagcggtttaatgctgcctttttattgtgcagtgaatagatagcaggtatcctaatttcattaagcaatctggaagatgaataaaaattgaaggacaaacacgtataatacataaaaaagattaactctacagttaatcttttttattcagaagaaaatatcctaactttgaaactaaatacaaagtaaaagcaatcattacagttctagatattacaattccatgaatagctagatcatatccagcaggtatcaacgcatttgtattacacataaaatatatagatattagaagtgctacaataactaaaatcattccaaaaagacttgttttttcatatttcataccaatttccacccttattaaagttaggtttaaacaaaagagctgaagaaacgaactatgaccagtatgctccaaggaaaaccgccagacaatgctggcggctttttgctgcttcgtttatttattaacagagatcgtaacgttatttcctgcaactgaaacctttgcgaaatcc

SEQ ID NO: 5 - DNA, 59 bp, Artificial Sequence
  multiple cloning site
agcttgcatgcctgcaggtcgactctagaggatcccccgggggtaccgagctcgaattc

SEQ ID NO: 6 - DNA, 1910 bp, Artificial Sequence
  thyA vector
cagatcatataaggaatgaaccgctgccaaatatcataaaaaagttgttaatgatcaaatgaatgaaattagagagaatttattttaaagaaagcccaattgcacatggacaaatgacaattgatattgaggtaaaatacaatgcattaaatcagaaaaagctattggaggatttaatgtgtttagacaatttccaatttggtatacacaaacacctgactatttgaatttttatgtaccgcaatatcaaaccatttcgtataatcctcaacaatgttatcaacggtgtatgtaccaaactggcggtaactatgagctatgtgacagactatgttatggagaaatacaggtgtaaaagagggggattaactcctctttaaacacacagtgagtggaataagatcctcactttatctgcaagtgcttagtatttgcgataatattgcattcgtaataaattatgcttagcaactgaaaatgaaagaaggatatgaatagtcatgacgcaattcgataaacaatacaattcaattataaaggatattatcaataatggaatctcagacgaagagtttgatgtaagaaccaagtgggactcagatggaacaccggcacatactctaagtgtaatcagtaagcaaatgagattcgacaactcagaggttccgattttaacgacaaaaaaggttgcctggaaaacagccattaaagagttgctctggatttggcagctgaaatctaatgatgttaatgatttaaacatgatgggcgtccatatttgggatcagtggaaacaagaagacggaaccatcggacatgcatatggatttcagctggggaagaaaaacagaagtctaaatggagaaaaagtggatcaggtagactatcttcttcatcaataagcttgcatgcctgcaggtcgactctagaggatcccccgggggtaccgagctcgaattctgaagaacaatccatcttcacgcagacacattacaatgctgtggaatcctgatgaattagacgcaatggccttaacgccatgtgtatacgagacacaatggtacgttaaacatgggaaactccaccttgaggtaagagcacggagcaatgatatggcattgggaaatccattcaatgtattccagtataatgtgttgcagcgcatgattgctcaagtgactggttatgagcttggtgaatatatctttaacattggggattgccatgtgtacacacgtcatatagacaatttgaaaattcaaatggaaagagaacagtttgaagcacctgaactatggatcaatcctgaagtgaaagatttttatgactttaccattgatgatttcaagttaatcaactataaacatggggacaagcttttatttgaggtagcggtttaatgctgcctttttattgtgcagtgaatagatagcaggtatcctaatttcattaagcaatctggaagatgaataaaaattgaaggacaaacacgtataatacataaaaaagattaactctacagttaatcttttttattcagaagaaaatatcctaactttgaaactaaatacaaagtaaaagcaatcattacagttctagatattacaattccatgaatagctagatcatatccagcaggtatcaacgcatttgtattacacataaaatatatagatattagaagtgctacaataactaaaatcattccaaaaagacttgttttttcatatttcataccaatttccacccttattaaagttaggtttaaacaaaagagctgaagaaacgaactatgaccagtatgctccaaggaaaaccgccagacaatgctggcggctttttgctgcttcgtttatttattaacagagatcgtaacgttatttcctgcaactgaaacctttgcgaaatcc

SEQ ID NO: 7 - DNA, 900 bp, Artificial Sequence
  left arm of thyB
ccaaatctgccgctcagtgtttgcatggagaatgtagaaaaagtcctgaacaaacgtgaaattattcatgctgttttgacaggccttgcactcgatcagcttgcagaacagaaacttctccccgaaccgctgcagcaccttgttgaaacggatgaaccgctttacggcatagatgaaattatcccgctttcaatcgttaatgtgtacgggtcgatcggtttgaccaatttcggttatttggataaagagaagattggaattattaaggaacttgatgaaagtccagacggtattcacacctttttggatgatattgtggcagctcttgctgcagcagcggcgagcagaattgcacatacgcatcaggatctgcaagatgaagaaaaagaacaggatgaaaagcctgtcgtcagctgactataaaaaaatcatttctgggttcagaaatgattttttattgtgttacactactagaagactacttttaaaggatgaaaaaaatgaaacagtataaggatttctgcagacatgttttagagcatggtgagaaaaagggagaccggactgggaccggaacaatcagcactttcggatatcaaatgagatttaatttacgggaaggctttccgatgctcaccactaaaaaactccactttaaatcaattgcgcatgaactgctgtggttcttaaaaggagatacgaatgtacgctatctgcaggaaaacggagtgcgaatctggaatgagtgggctgatgaaaacggtgaacttggacctgtatatggctcccaatggcgttcttggcggggagctgatggagaaaccattgatcaaatttcccgtcttattgaagatattaaaacaaatccgaactccagacgcttaatcgtcagcgcctgga

SEQ ID NO: 8 - DNA, 1095 bp, Artificial Sequence
  right arm of thyB
atgttggtgaaattgataaaatggcgttgccgccgtgccattgcctgttccaattctatgtgtctgacggcaagctgtcctgtcagctgtatcagcgctctgccgatgttttcttaggtgtgccgtttaatattgcatcttatgccctcctaaccatgatcattgctcatgtgactgggcttgaaccgggcgagttcatccatacgtttggtgatgttcatatttaccaaaatcatattgaacaagtcaatttgcagctggaaagagatgttagaccgcttccgcagcttcgtttcgccagaaaggttgattctatttttaactttgcatttgaggactttatcatcgaggattatgatccgcatcctcatataaaaggggcggtcagcgtatgatttcattcatttttgcgatggatgccaacaggcttatcggcaaagacaatgatttgccgtggcatttgcccaatgatcttgcatactttaagaaaataacatcgggccattcaatcattatgggccggaaaacatttgaatcgatcggacgtccgcttccaaatcggaaaaatattgtcgttacctcagcgccggattcagaatttcagggatgcacggttgtcagttcattaaaggatgtactggacatttgttcaggccctgaagaatgctttgtgatcggaggggctcagctctatacggacctgttcccttatgcggacagactgtatatgacgaaaattcatcacgagtttgagggtgaccgtcactttcctgaatttgatgaatccaattggaagctggtttcttctgagcaggggaccaaagacgaaaaaaacccgtatgattacgaatttctaatgtatgaaaaaaagaaatcttctaaagcgggaggattttaattggttcgctacagccttctagtggtttatattgtgtatatgctgttaaaaaatatgaaacaattatttaatcaaacaatgctcgatccccgtctgtcatacaaaaaacagatggctcttgtgtacgaacagccaaaggcgtttttagaaggctgtatcggcatctccggttcagttgtgacgatccatcagccaga

SEQ ID NO: 9 - DNA, 62 bp, Artificial Sequence
  multiple cloning site
aagcttgcatgcctgcaggtcgactctagaggatcccccgggggtaccgagctcgaattctg

SEQ ID NO: 10 - DNA, 2057 bp, Artificial Sequence
  thyB vector
ccaaatctgccgctcagtgtttgcatggagaatgtagaaaaagtcctgaacaaacgtgaaattattcatgctgttttgacaggccttgcactcgatcagcttgcagaacagaaacttctccccgaaccgctgcagcaccttgttgaaacggatgaaccgctttacggcatagatgaaattatcccgctttcaatcgttaatgtgtacgggtcgatcggtttgaccaatttcggttatttggataaagagaagattggaattattaaggaacttgatgaaagtccagacggtattcacacctttttggatgatattgtggcagctcttgctgcagcagcggcgagcagaattgcacatacgcatcaggatctgcaagatgaagaaaaagaacaggatgaaaagcctgtcgtcagctgactataaaaaaatcatttctgggttcagaaatgattttttattgtgttacactactagaagactacttttaaaggatgaaaaaaatgaaacagtataaggatttctgcagacatgttttagagcatggtgagaaaaagggagaccggactgggaccggaacaatcagcactttcggatatcaaatgagatttaatttacgggaaggctttccgatgctcaccactaaaaaactccactttaaatcaattgcgcatgaactgctgtggttcttaaaaggagatacgaatgtacgctatctgcaggaaaacggagtgcgaatctggaatgagtgggctgatgaaaacggtgaacttggacctgtatatggctcccaatggcgttcttggcggggagctgatggagaaaccattgatcaaatttcccgtcttattgaagatattaaaacaaatccgaactccagacgcttaatcgtcagcgcctggaaagcttgcatgcctgcaggtcgactctagaggatcccccgggggtaccgagctcgaattctgatgttggtgaaattgataaaatggcgttgccgccgtgccattgcctgttccaattctatgtgtctgacggcaagctgtcctgtcagctgtatcagcgctctgccgatgttttcttaggtgtgccgtttaatattgcatcttatgccctcctaaccatgatcattgctcatgtgactgggcttgaaccgggcgagttcatccatacgtttggtgatgttcatatttaccaaaatcatattgaacaagtcaatttgcagctggaaagagatgttagaccgcttccgcagcttcgtttcgccagaaaggttgattctatttttaactttgcatttgaggactttatcatcgaggattatgatccgcatcctcatataaaaggggcggtcagcgtatgatttcattcatttttgcgatggatgccaacaggcttatcggcaaagacaatgatttgccgtggcatttgcccaatgatcttgcatactttaagaaaataacatcgggccattcaatcattatgggccggaaaacatttgaatcgatcggacgtccgcttccaaatcggaaaaatattgtcgttacctcagcgccggattcagaatttcagggatgcacggttgtcagttcattaaaggatgtactggacatttgttcaggccctgaagaatgctttgtgatcggaggggctcagctctatacggacctgttcccttatgcggacagactgtatatgacgaaaattcatcacgagtttgagggtgaccgtcactttcctgaatttgatgaatccaattggaagctggtttcttctgagcaggggaccaaagacgaaaaaaacccgtatgattacgaatttctaatgtatgaaaaaaagaaatcttctaaagcgggaggattttaattggttcgctacagccttctagtggtttatattgtgtatatgctgttaaaaaatatgaaacaattatttaatcaaacaatgctcgatccccgtctgtcatacaaaaaacagatggctcttgtgtacgaacagccaaaggcgtttttagaaggctgtatcggcatctccggttcagttgtgacgatccatcagccaga

SEQ ID NO: 11 - protein, 272 aa, Artificial Sequence
  CotC-VP26
MGYYKKYKEEYYTVKKTYYKKYYEYDKKDYDCDYDKKYDDYDKKYYDHDKKDYDYVVEYKKHKKHYKLMEFGNLTNLDVAIIAILSIAIIALIVIMVIMIVFNTRVGRSVVANYDQMMRVPIQRRAKVMSIRGERSYNTPLGKVAMKNGLSDKDMKDVSADLVISTVTAPRTDPAGTGAENSNMTLKILNNTGVDLLINDITVRPTVIAGNIKGNTMSNTYFSSKDIKSSSSKITLIDVCSKFEDGAAFEATMNIGFTSKNVIDIKDEIKKK

SEQ ID NO: 12 - protein, 481 aa, Artificial Sequence
  CotB-VP28
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKLMDLSFTLSVVSAILAITAVIAVFIVIFRYHNTVTKTIETHTGNIETNMDENLRIPVTAEVGSGYFKMTDVSFDSDTLGKIKIRNGKSDAQMKEEDADLVITPVEGRALEVTVGQNLTFEGTFKVWNNTSRKINITGMQMVPKINPSKAFVGSSNTSSFTPVSIDEDEVGTFVCGTTFGAPIAATAGGNLFDMYVHVTYSGTETE

SEQ ID NO: 13 - protein, 597 aa, Artificial Sequence
  CotB-TcdA26-39
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKLASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAP

SEQ ID NO: 14 - protein, 388 aa, Artificial Sequence
  CotC-TcdA26-39
MGYYKKYKEEYYTVKKTYYKKYYEYDKKDYDCDYDKKYDDYDKKYYDHDKKDYDYVVEYKKHKKHYKLASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAP

SEQ ID NO: 15 - protein, 436 aa, Artificial Sequence
  CotB-SA
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSIQDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 16 - protein, 552 aa, Artificial Sequence
  CotB-AprE
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKLAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 17 - protein, 903 aa, Artificial Sequence
  CotB-AmyE
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKLETANKSNEVAASSVKNGTILHAWNWSFNTLTQNMKEIRDAGYAAIQTSPINQVKEGNQGDKSMRNWYWLYQPTSYQIGNRYLGTEQEFKDMCAAAEKYGLKVIVDAVINHTTSDYAAISDEIKRIPNWTHGNTQIKNWSDRWDVTQNSLLGLYDWNTQNTEVQTYLKGFLERALNDGADGFRYDAAKHIELPDDGNYGSRFWPNITNTSAEFQYGEILQDSASRDTAYANYMNVTASNYGHSIRSALKNRNLSVSNISHYASDVSADKLVTWVESHDTYANDEEESTWMSDDDIRLGWAVIGSRSGSTPLFFSRPEGGGNGVRFPGKSQIGDRGSALFKDQAITAVNQFHNVMAGQPEELSNPNGNNQIFMNQRGSKGVVLANAGSSSVTVNTSTKLPDGRYDNRAGAGSFQVANGKLTGTINARSAAVLYSDDIGNAPQVFLENYQTGAVHSFNDQLTVTLRANAKTTKAVYQINNGQQTAFKDGDRLTIGKGDPIGTTYNIRLTGTNGEGAERTQEYTFVKKDPAQTNIIGYQNPDHWGQVNAYIYKHDGGGAIELTGSWPGKAMTKNANGIYTLTLPANADTANAKVIFNNGSAQVPGQNQPGFDYVQNGLYNNSGLNGYLPH

SEQ ID NO: 18 - protein, 279 aa, Artificial Sequence
  open reading frame of thyA
MTQFDKQYNSIIKDIINNGISDEEFDVRTKWDSDGTPAHTLSVISKQMRFDNSEVPILTTKKVAWKTAIKELLWIWQLKSNDVNDLNMMGVHIWDQWKQEDGTIGHAYGFQLGKKNRSLNGEKVDQVDYLLHQLKNNPSSRRHITMLWNPDELDAMALTPCVYETQWYVKHGKLHLEVRARSNDMALGNPFNVFQYNVLQRMIAQVTGYELGEYIFNIGDCHVYTRHIDNLKIQMEREQFEAPELWINPEVKDFYDFTIDDFKLINYKHGDKLLFEVAV

SEQ ID NO: 19 - protein, 264 aa, Artificial Sequence
  open reading frame of thyB
MKQYKDFCRHVLEHGEKKGDRTGTGTISTFGYQMRFNLREGFPMLTTKKLHFKSIAHELLWFLKGDTNVRYLQENGVRIWNEWADENGELGPVYGSQWRSWRGADGETIDQISRLIEDIKTNPNSRRLIVSAWNVGEIDKMALPPCHCLFQFYVSDGKLSCQLYQRSADVFLGVPFNIASYALLTMIIAHVTGLEPGEFIHTFGDVHIYQNHIEQVNLQLERDVRPLPQLRFARKVDSIFNFAFEDFIIEDYDPHPHIKGAVSV

(19) United States
(12) Patent Application Publication
(10) Pub. No.: US 2020/0354727 A1
(43) Pub. Date: Nov. 12, 2020

(54) MODIFIED BACTERIAL SPORES

(71) Applicant: SPOREGEN LIMITED, Egham Surrey (GB)

(72) Inventor: Simon CUTTING, Egham Surrey (GB)

(21) Appl. No.: 16/760,827

(22) PCT Filed: Nov. 1, 2018
(86) PCT No.: PCT/GB2018/053178

§ 371 Date: Apr. 30, 2020

(30) Foreign Application Priority Data
Nov. 2, 2017 (GB) 1718133.0

(51) Int. Cl. C12N 15/75; C12N 9/10; C12N 3/00; C12N 1/20

(52) U.S. Cl. CPC C12N 15/75; C12N 9/1007; C12N 2500/40; C12N 3/00; C12N 1/20; C12Y 201/01045

(57) ABSTRACT

The present invention relates to modified bacterial spores, and particularly, although not exclusively, to modified bacterial spores which comprise one or more heterologous genes, but without the introduction of antibiotic resistance genes. The invention extends to methods for producing such modified bacterial spores, vectors and kits for introducing heterologous genes in a spore.

### CROSS-REFERENCE

This application is a 371 National Stage filing and claims the benefit under 35 U.S.C. § 120 to International Application No. PCT/GB2018/053178, filed Nov. 1, 2018, which claims priority to Great Britain Application No. 1718133.0, filed Nov. 2, 2017, each of which is incorporated herein by reference in its entirety.

### SEQUENCE LISTING

This instant application contains a Sequence Listing which has been submitted in ASCII format via EFS-Web and is hereby incorporated by reference in its entirety. Said ASCII copy, created on Apr. 23, 2020, is named 06245-006US1_Sequence_Listing and is 50 KB in size.

The present invention relates to modified bacterial spores, and particularly, although not exclusively, to modified bacterial spores which comprise one or more heterologous genes, but without the introduction of antibiotic resistance genes. The invention extends to methods for producing such modified bacterial spores, vectors and kits for introducing heterologous genes in a spore-forming bacterium.

Bacillus endospores, or spores, are dormant entities with well-known resistance properties including heat stability, exposure to noxious chemicals and desiccation (1). As such, spores offer a number of applications. First and foremost has been their use as mucosal vaccines where antigens can be displayed on the spore surface by fusion to a coat protein anchor (2). Immunization of recombinant spores by a mucosal route (oral, sub-lingual or nasal) has shown promising results and in some cases levels of protection that could be efficacious in humans or animals (3-5). In the case of the latter, the ability to incorporate a vaccine in feed is particularly attractive and for some animals, arguably, the only way to vaccinate. For example, vaccinating farmed shrimp against viral pathogens, for example, White Spot Syndrome Virus (WSSV) where recombinant spores expressing the WSSV VP26 and VP28 envelope proteins have been shown to confer protection in shrimps (6-9).

A number of Bacillus species are considered ‘food grade’, that is, safe for human consumption and are designated as QPS (Qualified Presumption of Safety as defined by EFSA, European Food Safety Authority) (10). In the USA, some strains carry GRAS (Generally Recognized As Safe as defined by the Food and Drug Administration) status. QPS and GRAS status has supported the use of a number of Bacillus strains, including those of B. subtilis, as probiotics in both human and animal feeds (11). The concept of recombinant probiotics (12) is conceptually, a logical next step forward in exploiting the beneficial properties of Bacillus (13).

In addition to vaccines, spores have been shown to facilitate expression of enzymes on the spore surface, for example the animal feed enzyme phytase (14). The ability to use probiotic bacteria that deliver enzymes negates the need to purify enzymes and could offer significant advantages to industry. Finally, streptavidin has been expressed on B. subtilis spores enabling monoclonal antibodies to be conjugated to the spore surface and the targeting of spores loaded with anti-cancer drugs to cancer cells (15).

Despite the potential utility of Bacillus spores for industrial applications, there remain a number of obstacles regarding the deliberate release of genetically modified Bacillus. First, is the use of antibiotic resistance (Ab^{R}) genes used in the genetic engineering of stable recombinant strains. Regarding Ab^{R} the majority of procedures requiring insertion of heterologous DNA into B. subtilis require ectopic insertion using an Ab^{R} gene for positive selection (16). Plasmid vectors carrying chloramphenicol or erythromycin resistance genes are typically used for ectopic insertion at a genetic locus that is redundant for cell growth, typically the alpha amylase (amyE) or threonine C (thrC) genes (17). The potential risk of Ab^{R} gene transfer following release is recognised and at least two systems in B. subtilis have now been described that enable insertion of heterologous genes without introduction of an Ab^{R} gene (18,19).

The second, and most challenging, hurdle is the ultimate fate of recombinant spores once released, since spores have been shown to survive for millions of years as viable entities (20). The soil is normally enriched with dormant spores (21) and their intrinsic robustness makes it difficult to argue that they would not persist after deliberate release. One approach might be to construct germination deficient spores but, at best, the germination rate can be reduced to 0.0015% (22) which is unlikely to satisfy regulatory authorities.

Therefore, there is a need for improved methods of introducing heterologous genes into spore-forming bacteria without the introduction of antibiotic resistance genes.

The inventors have adopted the approach of thymine starvation resulting in a so-called ‘thymine-less death’ (23-25). The majority of prokaryotes are known to carry a single thymidylate synthase enzyme that is encoded by the thyA gene and prokaryotes carrying a mutated thymidylate synthase gene are unable to grow in low concentrations of thymidine or thymine resulting in cell death. Thymine starvation therefore is different from the biostatic effects commonly found with deprivation of other nutrients. Thymine-less death has been documented for B. subtilis (26) and the bacterium is unique in that it carries two thymidylate synthase enzymes encoded by the thyA and thyB genes (27).

However, the concept of thymine-less death, despite being known since 1954, has never been successfully applied to spore-forming bacteria for the purposes of introducing heterologous genes without the introduction of antibiotic resistance genes.

The inventors have successfully adopted the principles of a ‘thymine-less death’ to exploit spore-forming bacteria, such as Bacillus, for the introduction of heterologous genes without the introduction of antibiotic resistance genes. As recombinant spores, should they germinate, they will die immediately and fail to survive or proliferate (i.e., grow) in the environment due to the lack of (or only low concentrations of) thymine or thymidine. The approach developed is particularly novel since it is a two-step gene integration process enabling positive selection on successively higher levels of trimethoprim, but without the introduction of any antibiotic resistance genes.

Thus, according to a first aspect of the invention, there is provided a process for introducing at least one heterologous gene into a spore-forming bacterium and rendering the bacterium unable to proliferate in the absence of thymine or thymidine, the process comprising:
(i) introducing a heterologous gene into a thymidylate synthase gene within a spore-forming bacterium; and
(ii) growing said spore-forming bacterium in the presence of trimethoprim.

In a preferred embodiment, and advantageously, the process operates without the introduction of an antibiotic resistance gene, and therefore overcomes the problems discussed above.

In a preferred embodiment, the method further comprises:
(iii) forming a bacterial spore from said spore-forming bacterium.

It will be appreciated that the bacterial spore may or may not germinate, but if it does germinate, it is unable to grow out of that germinated spore and proliferate.

Advantageously, the inventor has devised an elegant way to inactivate all thymidylate synthases in a bacterium, for example in embodiments in which the bacterium carries two or more thy genes, or where the bacterium carries a thyA and a thyB gene (e.g., Bacillus spp.), or where the inactivation of thyA is unable to affect the growth of the bacterium under normal physiological conditions.

Hence, in a preferred embodiment, one or more heterologous genes is introduced into each thymidylate synthase gene present within the bacterium. For instance, in one embodiment, the spore-forming bacterium may be a Bacillus Spp. (which is unique in that it is known to have two thymidylate synthase genes, thyA and thyB). Preferably, a heterologous gene is introduced into each of the two thymidylate synthase genes, for example thyA and thyB of Bacillus spp. The Bacillus spp. bacterium may be B. subtilis, B. cereus, B. pumilus, B. amyloliquefaeceans, B. coagulans, B. clausii, B. licheniformis or B. megaterium. Most preferably, however, the Bacillus spp. bacterium is B. subtilis.

It should be appreciated that inactivation of thyA only in Bacillus will not induce a thymineless death, and will not damage or prevent growth under normal physiological conditions.

In another embodiment, the spore-forming bacterium may be Clostridium Spp. (which is known to have one thymidylate synthase gene). Preferably, in this embodiment, a heterologous gene is introduced into the thymidylate synthase gene. The Clostridium spp. bacterium may be C. difficile, C. perfringens, C. tetani, C. botulinum, C. acetobutylicum, C. cellulolyticum, C. novyi or C. thermocellum. Most preferably, the Clostridium spp. bacterium is Clostridium difficile.

In preferred embodiments of the process, the spore-forming bacterium is present in a bacterial culture.

Step (ii) of the process may be performed using any suitable vessel, such as a Petri dish or glass culture flask, containing any growth medium as disclosed herein. The growth medium may comprise trimethoprim at a concentration of 0.001 μg/ml to 20 μg/ml, 0.01 μg/ml to 15 μg/ml, 0.1 μg/ml to 10 μg/ml, 1 μg/ml to 5 μg/ml, 2 μg/ml to 4 μg/ml, or preferably approximately 3 μg/ml.

In a further embodiment, during step (ii), the bacterium may be grown in a medium comprising yeast extract (YE) at a concentration lower than 2 mg/ml, or any concentration disclosed herein. In an embodiment, the medium contains no YE.

In an embodiment, during step (ii), the bacterium may be grown in a medium comprising thymine or thymidine at any concentration disclosed herein, for instance approximately 50 μg/ml.

In an embodiment, during step (ii), the bacterium may be grown in a medium comprising trimethoprim at any concentration disclosed herein, for instance approximately 3 μg/ml.

In an embodiment, during step (ii), the bacterium may be grown in a medium comprising casamino acids (CAA) at any concentration disclosed herein, for instance approximately 0.2% (w/v).

In an embodiment, during step (ii), the bacterium may be grown in a medium supplemented with at least one amino acid or any amino acid mixture as disclosed herein.

In an embodiment, cells carrying a gene insertion may be verified by the inability to grow unless supplemented with thymine or thymidine. A further or alternative verification is to amplify, by PCR, the presence of the chimeric genes from transformants.

In a most preferred embodiment, the invention provides a cloning system suitable for biological containment of genetically modified Bacillus Spp., such as B. subtilis.

Hence, according to this embodiment of the invention, there is provided a process for introducing at least one heterologous gene into a Bacillus Spp. and rendering the bacterium unable to proliferate in the absence of thymine or thymidine, the process comprising:
(i) introducing a heterologous gene into the thyA gene and the thyB gene in a Bacillus Spp.; and
(ii) growing said Bacillus Spp. in the presence of trimethoprim.

In a preferred embodiment, the heterologous gene is introduced into one of the thy genes, and then subsequently into the other thy gene. Preferably, therefore, there is provided a process for introducing at least one heterologous gene into a Bacillus Spp. and rendering the bacterium unable to proliferate in the absence of thymine or thymidine, the process comprising:
(i) introducing a heterologous gene into either the thyA gene or the thyB gene in a Bacillus Spp.;
(ii) growing said Bacillus Spp. in the presence of trimethoprim;
(iii) introducing a heterologous gene into the other of the thyA gene or the thyB gene in said Bacillus Spp.; and
(iv) growing said Bacillus Spp. in the presence of trimethoprim.

In a preferred embodiment, the process further comprises:
(v) forming a bacterial spore from said Bacillus Spp.

It will be appreciated that the bacterial spore may or may not germinate, but if it does germinate, it is unable to grow out of that germinated spore and proliferate. In a preferred embodiment, the process operates without the introduction of an antibiotic resistance gene. In a preferred embodiment, step (i) is the introduction of a heterologous gene into the thyA gene in a Bacillus Spp. and step (iii) is the introduction of a heterologous gene into the thyB gene in a Bacillus Spp.

In a preferred embodiment, the Bacillus Spp. is B. subtilis. In preferred embodiments, the Bacillus Spp. or B. subtilis is present in a bacterial culture.

In one embodiment, the open reading frame of thyA may be encoded by the nucleic acid sequence provided herein as SEQ ID NO: 1, as follows:

[SEQ ID NO: 1]
ATGACGCAATTCGATAAACAATACAATTCAATTATAAAGGATATTA
TCAATAATGGAATCTCAGACGAAGAGTTTGATGTAAGAACCAAGTG
GGACTCAGATGGAACACCGGCACATACTCTAAGTGTAATCAGTAAG
CAAATGAGATTCGACAACTCAGAGGTTCCGATTTTAACGACAAAAA
AGGTTGCCTGGAAAACAGCCATTAAAGAGTTGCTCTGGATTTGGCA
GCTGAAATCTAATGATGTTAATGATTTAAACATGATGGGCGTCCAT
ATTTGGGATCAGTGGAAACAAGAAGACGGAACCATCGGACATGCAT
ATGGATTTCAGCTGGGGAAGAAAAACAGAAGTCTAAATGGAGAAAA
AGTGGATCAGGTAGACTATCTTCTTCATCAATTGAAGAACAATCCA
TCTTCACGCAGACACATTACAATGCTGTGGAATCCTGATGAATTAG
ACGCAATGGCCTTAACGCCATGTGTATACGAGACACAATGGTACGT
TAAACATGGGAAACTCCACCTTGAGGTAAGAGCACGGAGCAATGAT
ATGGCATTGGGAAATCCATTCAATGTATTCCAGTATAATGTGTTGC
AGCGCATGATTGCTCAAGTGACTGGTTATGAGCTTGGTGAATATAT
CTTTAACATTGGGGATTGCCATGTGTACACACGTCATATAGACAAT
TTGAAAATTCAAATGGAAAGAGAACAGTTTGAAGCACCTGAACTAT
GGATCAATCCTGAAGTGAAAGATTTTTATGACTTTACCATTGATGA
TTTCAAGTTAATCAACTATAAACATGGGGACAAGCTTTTATTTGAG
GTAGCGGTTTAA

Preferably, therefore, the process comprises introducing a heterologous gene into a nucleic acid sequence substantially as set out in SEQ ID NO: 1, or a fragment or variant thereof, within Bacillus Spp. In an alternative embodiment, the processes may comprise introducing a heterologous gene into Bacillus Spp. not into SEQ ID NO: 1 or a fragment or variant thereof, but such that the expression of SEQ ID NO: 1 is disrupted or reduced.

In one embodiment, the open reading frame of thyA may be encoded by a nucleic acid sequence which encodes an amino acid sequence provided herein as SEQ ID NO: 18, as follows:

[SEQ ID NO: 18
MTQFDKQYNSIIKDIINNGISDEEFDVRTKWDSDGTPAHTLSVISK
QMRFDNSEVPILTTKKVAWKTAIKELLWIWQLKSNDVNDLNMPIGV
HIWDQWKQEDGTIGHAYGFQLGKKNRSLNGEKVDQVDYLLHQLKNN
PSSRRHITMLWNPDELDAMALTPCVYETQWYVKHGKLHLEVRARSN
DMALGNPFNVFQYNVLQRMIAQVTGYELGEYIFNIGDCHVYTRHID
NLKIQMEREQFEAPELWINPEVKDFYDFTIDDFKLINYKHGDKLLF
EVAV

SEQ ID NO: 1 and 18 are examples from B. subtilis, and thus variants of these sequences include the relevant thyA genes from all Bacillus Spp. It is important to note that the invention is applicable to any thyA of a Bacillus Spp. and that SEQ ID NO: 1 and 18 are intended to be illustrative and not limiting.

In one embodiment, the open reading frame of thyB may be encoded by the nucleic acid sequence provided herein as SEQ ID NO: 2, as follows:

[SEQ ID NO: 2]
ATGAAACAGTATAAGGATTTCTGCAGACATGTTTTAGAGCATGGTG
AGAAAAAGGGAGACCGGACTGGGACCGGAACAATCAGCACTTTCGG
ATATCAAATGAGATTTAATTTACGGGAAGGCTTTCCGATGCTCACC
ACTAAAAAACTCCACTTTAAATCAATTGCGCATGAACTGCTGTGGT
TCTTAAAAGGAGATACGAATGTACGCTATCTGCAGGAAAACGGAGT
GCGAATCTGGAATGAGTGGGCTGATGAAAACGGTGAACTTGGACCT
GTATATGGCTCCCAATGGCGTTCTTGGCGGGGAGCTGATGGAGAAA
CCATTGATCAAATTTCCCGTCTTATTGAAGATATTAAAACAAATCC
GAACTCCAGACGCTTAATCGTCAGCGCCTGGAATGTTGGTGAAATT
GATAAAATGGCGTTGCCGCCGTGCCATTGCCTGTTCCAATTCTATG
TGTCTGACGGCAAGCTGTCCTGTCAGCTGTATCAGCGCTCTGCCGA
TGTTTTCTTAGGTGTGCCGTTTAATATTGCATCTTATGCCCTCCTA
ACCATGATCATTGCTCATGTGACTGGGCTTGAACCGGGCGAGTTCA
TCCATACGTTTGGTGATGTTCATATTTACCAAAATCATATTGAACA
AGTCAATTTGCAGCTGGAAAGAGATGTTAGACCGCTTCCGCAGCTT
CGTTTCGCCAGAAAGGTTGATTCTATTTTTAACTTTGCATTTGAGG
ACTTTATCATCGAGGATTATGATCCGCATCCTCATATAAAAGGGGC
GGTCAGCGTATGA

Preferably, therefore, the process comprises introducing a heterologous gene into a nucleic acid sequence substantially as set out in SEQ ID NO: 2, or a fragment or variant thereof, within Bacillus Spp. The processes may comprise introducing a heterologous gene into Bacillus Spp. not into SEQ ID NO: 2 or a fragment or variant thereof, but such that the expression from SEQ ID NO: 2 is disrupted or reduced.

In one embodiment, the open reading frame of thyB may be encoded by a nucleic acid sequence which encodes an amino acid sequence provided herein as SEQ ID NO: 19, as follows:

[SEQ ID NO: 19]
MKQYKDFCRHVLEHGEKKGDRTGTGTISTFGYQMRFNLREGFPMLT
IKKLHFKSIAHELLWFLKGDINVRYLQENGVRIWNEWADENGELGP
VYGSQWRSWRGADGETIDQISRLIEDIKTNPNSRRLIVSAWNVGEI
DKMALPPCHCLFQFYVSDGKLSCQLYQRSADVFLGVPFNIASYALL
TMIIAHVTGLEPGEFIHTFGDVHIYQNHIEQVNLQLERDVRPLPQL
RFARKVDSIFNFAFEDFIIEDYDPHPHIKGAVSV

SEQ ID NO: 2 and 19 are examples from B. subtilis, and thus variants of these sequences include the relevant thyB genes from all Bacillus Spp. It is important to note that the invention is applicable to any thyB of a Bacillus Spp. and that SEQ ID NO: 2 and 19 are intended to be illustrative and not limiting.

In an embodiment, step (i) may be performed by the transformation of Bacillus Spp. with a plasmid, which may be linearised, which may comprise at least a portion of the left arm of a Bacillus Spp. thyA gene, a heterologous gene, and at least a portion of the right arm of a Bacillus Spp. thyA gene. Step (i) may be performed with any vector as disclosed herein. Transformation may be carried out using electroporation.

Step (ii) and/or Step (iv) may be performed using any suitable vessel, such as a Petri dish or a cell culture flask, containing any growth medium as disclosed herein. In an embodiment, during step (ii) and/or step (iv), the Bacillus Spp. may be grown in a medium comprising yeast extract (YE) at a concentration lower than 2 mg/ml. In a preferred embodiment, the medium has YE at a concentration lower than 1.5 mg/ml, 1 mg/ml, 0.5 mg/ml, 0.25 mg/ml, 0.125 mg/ml, 0.1 mg/ml, 0.01 mg/ml, 0.001 mg/ml, 0.0001 mg/ml, or 0 mg/ml. In an embodiment, the medium in which the Bacillus Spp. is grown contains no YE.

In an embodiment, during step (ii) and/or step (iv), the Bacillus Spp. may be grown in a medium comprising thymine or thymidine. In a preferred embodiment, the thymine or thymidine may be present between 0.1 μg/ml and 1000 μg/ml, 1 μg/ml and 500 μg/ml, less than or equal to 300 μg/ml, 10 μg/ml and 200 μg/ml, 20 μg/ml and 100 μg/ml, 30 μg/ml and 80 μg/ml, 35 μg/ml and 65 μg/ml, 45 μg/ml and 55 μg/ml, or preferably approximately 50 μg/ml.

In an embodiment, during step (ii) and/or step (iv), the growth medium is any suitable growth medium, for instance Spizizen's Minimal Medium (SMM).

In an embodiment, the amount of trimethoprim present in step (iv) is higher than the amount of trimethoprim present in step (ii).

In an embodiment, during step (ii), the medium may comprise trimethoprim at a concentration of 0.001 μg/ml to 20 μg/ml, 0.01 μg/ml to 15 μg/ml, 0.1 μg/ml to 10 μg/ml, 1 μg/ml to 5 μg/ml, 2 μg/ml to 4 μg/ml, or preferably approximately 3 μg/ml.

In a preferred embodiment, the concentration of trimethoprim is lower than the concentration of trimethoprim in step (iv).

In an embodiment, step (ii) may comprise or consist of 12 hours to 168 hours, 24 hours to 144 hours, 48 hours to 120 hours, 60 hours to 108 hours, or 72 hours to 96 hours of growth.

In an embodiment, cells carrying an insertion at the thyA locus may be verified by the ability to grow at 37° C. with or without thymine or thymidine but the inability to grow at 46° C. unless supplemented with thymine or thymidine. A further, or alternative, verification is to amplify, by PCR, the presence of the chimeric gene from transformants using primers annealing to the thyA sequences. In some embodiments, the colonies may be screened and separated into two types: i) large opaque colonies (2-3 mm after 72 h-96 h of growth), ii) translucent, smaller colonies (1 mm after 72 h-96 h of growth). In some embodiments, type (i) colonies may be selected.

In an embodiment, step (iii) may be performed by the transformation of Bacillus Spp. with a plasmid, which may be linearised, which may comprise at least a portion of the left arm of a Bacillus Spp. thyB gene, a heterologous gene, and at least a portion of the right arm of a Bacillus Spp. thyB gene. Step (iii) may be performed with any vector as disclosed herein. Transformation may be carried out using electroporation.

In an embodiment, the Bacillus Spp. may be prepared or made electro-competent using a method that involves culturing Bacillus Spp. in medium that contains yeast extract (YE) at a concentration lower than 2 mg/ml. In a preferred embodiment, the medium has YE at a concentration lower than 1.5 mg/ml, 1 mg/ml, 0.5 mg/ml, 0.25 mg/ml, 0.125 mg/ml, 0.1 mg/ml, 0.01 mg/ml, 0.001 mg/ml, 0.0001 mg/ml, or 0 mg/ml. In an embodiment, the medium in which the Bacillus Spp. is grown contains no YE. In a most preferred embodiment, the Bacillus Spp. is a strain carrying a thyA insertion as generated by steps (i) and (ii).

In an embodiment, YE is present at a concentration lower than 2 mg/ml, any concentration disclosed herein, or preferably not present in the media used at any stage of the process.

In an embodiment, step (iv) may be performed using medium which may comprise trimethoprim at a concentration of less than or equal to 80 μg/ml, less than or equal to 40 μg/ml, less than or equal to 30 μg/ml, 0.01 μg/ml to 30 μg/ml, 0.1 μg/ml to 20 μg/ml, 1 μg/ml to 15 μg/ml, 2 μg/ml to 10 μg/ml, 5 μg/ml to 7 μg/ml, or preferably approximately 6 μg/ml. In a preferred embodiment the concentration of trimethoprim is higher than the concentration of trimethoprim in step (ii).

Casamino acids (CAA) are produced by the acid hydrolysis of casein. The typical composition is as follows, although these numbers are approximate and variations in composition are acceptable for use with the invention: Ala 2.8%, Arg 3.6%, Asp 6.3%, Cystine 0.3%, Glu 21.1%, Gly 2.2%, His 2.7%, Ile 5.6%, Leu 8.4%, Lys 7.5%, Met 2.7%, Phe 4.6%, Pro 9.9%, Ser 5.6%, Thr 4.2%, Trp 1.1%, Tyr 6.1%, Val 5.0%. CAA may not contain tryptophan.

In an embodiment, during step (iv), the Bacillus Spp. may be grown in a medium comprising casamino acids (CAA). The medium may comprise CAA at 0.0001% to 5%, 0.001% to 2%, 0.01% to 1%, 0.05% to 0.5%, 0.1% to 0.3%, or preferably approximately 0.2% (w/v).

In an embodiment, during step (iv), the Bacillus Spp. may be grown in a medium supplemented with at least one amino acid. In other embodiments the medium may comprise any naturally occurring amino acid or amino acids, and optionally all naturally occurring amino acids or all naturally occurring amino acids except tryptophan. The medium may comprise serine, glycine, cysteine, methionine, any combination thereof, or all four amino acids. The medium may comprise any individual amino acid at a concentration equivalent to medium containing CAA at 0.0001% to 5%, 0.001% to 2%, 0.01% to 1%, 0.05% to 0.5%, 0.1% to 0.3%, or preferably approximately 0.2% (w/v). Thus, in a preferred embodiment, the medium may comprise serine, glycine, cysteine, and/or methionine at a concentration equivalent to medium containing CAA at approximately 0.2% (w/v). In another preferred embodiment, the medium may comprise the same amino acids as medium containing CAA at approximately 0.2% (w/v).

In an embodiment, step (iv) may comprise or consist of 6 hours to 144 hours, 12 hours to 120 hours, 24 hours to 96 hours, 36 hours to 72 hours, or approximately 48 hours of growth.

In an embodiment, cells carrying two insertions may be verified by the inability to grow at 37° C. or 46° C. unless supplemented with thymine or thymidine. A further or alternative verification is to amplify, by PCR, the presence of the chimeric genes from transformants using primers annealing to the thyA and thyB sequences.

Any heterologous gene may be used for the gene insertion into the or each thymidylate synthase gene. In particular embodiments, the heterologous genes are fused to genes encoding spore coat proteins, for instance CotA, CotB, CotC, CotD, CotE, CotF, or CotG of B. subtilis. The spore protein used may be chosen depending on the particular bacterium being modified, and the appropriate gene used. Appropriate spore coat proteins for use with Clostridium Spp. include CotA, CotB, CotC, CotD, CotE, CotF, CdeC, BcA1, BcA2, or BcA3 (53, 54, 55).

In some embodiments, a heterologous gene may be inserted into the thyA gene and the same heterologous gene may be inserted into the thyB gene. In other embodiments, a different heterologous gene may be inserted into the thyA gene from that inserted into the thyB gene.

In some embodiments, the resultant spore produced by the process may carry a heterologous protein for use in vaccination. The resultant spore may carry a heterologous protein which is displayed as an active enzyme. The resultant spore may carry a heterologous protein allowing the conjugation of proteins to the spore surface.

After generation and selection of a strain comprising the heterologous genes that are unable to proliferate in the absence of thymine or thymidine, the process may comprise cultivating the strain. The cultivation may be on any media comprising thymine or thymidine, YE at a concentration less than 2 mg/ml or absent, and at least one amino acid or any mixture as disclosed herein. The concentrations may be any as disclosed herein.

Thus, according to a particular embodiment of the invention, there is provided a process for introducing at least one heterologous gene into B. subtilis and rendering the B. subtilis unable to proliferate in the absence of thymine or thymidine, preferably wherein the process does not involve the introduction of an antibiotic resistance gene, comprising:
(i) introducing a heterologous gene into the thyA gene or thyB gene within B. subtilis;
(ii) growing said B. subtilis in the presence of trimethoprim and either thymine or thymidine, and YE at a concentration lower than 2 mg/ml, optionally wherein no YE is present;
(iii) introducing a heterologous gene into the other of the thyA gene or thyB gene within said B. subtilis;
(iv) growing said Bacillus Spp. in the presence of trimethoprim and either thymine or thymidine, and YE at a concentration lower than 2 mg/ml, optionally wherein no YE is present.

In a preferred embodiment, YE is present at a concentration lower than 2 mg/ml, preferably 0 mg/ml, during all steps following step (i). In a preferred embodiment, thymine or thymidine is present for all cultures following step (i). In a preferred embodiment, a heterologous gene is introduced into the thyA gene during step (i) and a heterologous gene is introduced into the thyB gene during step (iii).

Thus, according to a particular embodiment of the invention, there is provided a process for introducing at least one heterologous gene into B. subtilis and rendering the B. subtilis unable to proliferate in the absence of thymine or thymidine, preferably wherein the process does not involve the introduction of an antibiotic resistance gene, comprising:
(i) introducing a heterologous gene into the thyA gene or the thyB gene within B. subtilis;
(ii) growing said B. subtilis in the presence of trimethoprim, and in the presence of either thymine or thymidine;
(iii) introducing a heterologous gene into the other of the thyA gene or thyB gene within said B. subtilis;
(iv) growing said Bacillus Spp. in the presence of trimethoprim and either thymine or thymidine, and at least one amino acid, optionally wherein CAA is present.

In a preferred embodiment, thymine or thymidine is present for all cultures following step (i). In a preferred embodiment, a heterologous gene is introduced into the thyA gene during step (i) and a heterologous gene is introduced into the thyB gene during step (iii).

According to a second aspect of the invention, there is provided a bacterial spore obtained or obtainable by the process of the first aspect.

Preferably, the bacterial spore is Bacillus spp. and comprises non-functional thyA and thyB genes, preferably due to the introduction of heterologous genes therein.

In a third aspect, there is provided a bacterial spore comprising at least one heterologous gene and unable to proliferate in the absence of thymine or thymidine, wherein the bacterial spore is produced by a process comprising:
(i) introducing a heterologous gene into a thymidylate synthase gene within a spore-forming bacterium;
(ii) growing said spore-forming bacterium in the presence of trimethoprim; and
(iii) forming a bacterial spore from said spore-forming bacterium.

In a preferred embodiment, the bacterial spore does not comprise an antibiotic resistance gene.

In a preferred embodiment, the bacterial spore may be Bacillus Spp. The Bacillus spp. bacterium may be B. subtilis, B. cereus, B. pumilus, B. amyloliquefaeceans, B. coagulans, B. clausii, B. licheniformis or B. megaterium. Most preferably, the Bacillus spp. bacterium is B. subtilis. In another preferred embodiment, the bacterial spore may be Clostridium Spp. The Clostridium spp. bacterium may be C. difficile, C. perfringens, C. tetani, C. botulinum, C. acetobutylicum, C. cellulolyticum, C. novyi or C. thermocellum. Most preferably, the Clostridium spp. bacterium is Clostridium difficile.

In an embodiment, there is provided a bacterial spore comprising at least one heterologous gene and unable to proliferate in the absence of thymine or thymidine, wherein the bacterial spore is produced by any process as disclosed herein, or using any culture or conditions as disclosed.

According to a particular embodiment of the invention, there is provided a Bacillus Spp. spore comprising at least one heterologous gene and unable to proliferate in the absence of thymine or thymidine, wherein the Bacillus Spp. spore is produced by a process comprising:
(i) introducing a heterologous gene into the thyA gene or the thyB gene within a Bacillus Spp.;
(ii) growing said Bacillus Spp. in the presence of trimethoprim and in the presence of thymine or thymidine, optionally in the presence of YE at a concentration lower than 2 mg/ml, preferably wherein no YE is present;
(iii) introducing a heterologous gene into the other of the thyA gene or thyB gene within said Bacillus Spp.;
(iv) growing said Bacillus Spp. in the presence of trimethoprim, optionally at a higher concentration than in step (ii), and in the presence of thymine or thymidine, optionally in the presence of YE at a concentration lower than 2 mg/ml, preferably wherein no YE is present, optionally in the presence of at least one amino acid, optionally wherein CAA is present; and
(v) forming bacterial spores from said Bacillus Spp.

In a preferred embodiment, a heterologous gene is introduced into the thyA gene during step (i) and a heterologous gene is introduced into the thyB gene during step (iii).

According to a fourth aspect of the invention, there is provided a vector for use in introducing a heterologous gene into the thyA gene within a spore-forming bacterium.

In an embodiment, the vector may comprise at least a portion of the 5′ portion of a Bacillus Spp. thyA gene, a heterologous gene, and at least a portion of the 3′ portion of a Bacillus Spp. thyA gene.

In an embodiment, the vector may comprise the left and right arms of a Bacillus Spp. thyA gene. The arms may flank a multiple cloning site. For instance, the vector may include the left (e.g. 900 bp) and right (e.g. 950 bp) arms of the B. subtilis thyA gene.

The length of the flanking arms may be adjusted for the length of the heterologous sequence to be inserted. Longer flanking arms (e.g. ˜1500 bp) may be necessary for large inserts.

The vectors may comprise a heterologous gene inserted at the multiple cloning site.

The heterologous gene may be a chimeric gene comprising a spore coat protein fused to a heterologous gene. For instance, the spore coat protein may be CotB or CotC of B. subtilis. The vectors may comprise: the left arm of a Bacillus Spp. thyA gene, a spore coat protein, a multiple cloning site allowing insertion of a heterologous gene such that it is fused to the spore coat protein, and the right arm of a Bacillus Spp. thyA gene.

In one purely illustrative embodiment, the left arm of thyA may be encoded by the nucleic acid sequence provided herein as SEQ ID NO: 3 as follows:

[SEQ ID NO: 3]
CAGATCATATAAGGAATGAACCGCTGCCAAATATCATAAAAAAGTT
GTTAATGATCAAATGAATGAAATTAGAGAGAATTTATTTTAAAGAA
AGCCCAATTGCACATGGACAAATGACAATTGATATTGAGGTAAAAT
ACAATGCATTAAATCAGAAAAAGCTATTGGAGGATTTAATGTGTTT
AGACAATTTCCAATTTGGTATACACAAACACCTGACTATTTGAATT
TTTATGTACCGCAATATCAAACCATTTCGTATAATCCTCAACAATG
TTATCAACGGTGTATGTACCAAACTGGCGGTAACTATGAGCTATGT
GACAGACTATGTTATGGAGAAATACAGGTGTAAAAGAGGGGGATTA
ACTCCTCTTTAAACACACAGTGAGTGGAATAAGATCCTCACTTTAT
CTGCAAGTGCTTAGTATTTGCGATAATATTGCATTCGTAATAAATT
ATGCTTAGCAACTGAAAATGAAAGAAGGATATGAATAGTCATGACG
CAATTCGATAAACAATACAATTCAATTATAAAGGATATTATCAATA
ATGGAATCTCAGACGAAGAGTTTGATGTAAGAACCAAGTGGGACTC
AGATGGAACACCGGCACATACTCTAAGTGTAATCAGTAAGCAAATG
AGATTCGACAACTCAGAGGTTCCGATTTTAACGACAAAAAAGGTTG
CCTGGAAAACAGCCATTAAAGAGTTGCTCTGGATTTGGCAGCTGAA
ATCTAATGATGTTAATGATTTAAACATGATGGGCGTCCATATTTGG
GATCAGTGGAAACAAGAAGACGGAACCATCGGACATGCATATGGAT
TTCAGCTGGGGAAGAAAAACAGAAGTCTAAATGGAGAAAAAGTGGA
TCAGGTAGACTATCTTCTTCATCAAT

The bold sequence represents the thyA coding sequence, the non-bold sequence represents the upstream region. Preferably, therefore, the vector comprises a nucleic acid sequence substantially as set out in SEQ ID NO: 3, or a fragment or variant thereof.

In one purely illustrative embodiment, the right arm of thyA may be encoded by the nucleic acid sequence provided herein as SEQ ID NO: 4 as follows:

[SEQ ID NO: 4]
TGAAGAACAATCCATCTTCACGCAGACACATTACAATGCTGTGGAA
TCCTGATGAATTAGACGCAATGGCCTTAACGCCATGTGTATACGAG
ACACAATGGTACGTTAAACATGGGAAACTCCACCTTGAGGTAAGAG
CACGGAGCAATGATATGGCATTGGGAAATCCATTCAATGTATTCCA
GTATAATGTGTTGCAGCGCATGATTGCTCAAGTGACTGGTTATGAG
CTTGGTGAATATATCTTTAACATTGGGGATTGCCATGTGTACACAC
GTCATATAGACAATTTGAAAATTCAAATGGAAAGAGAACAGTTTGA
AGCACCTGAACTATGGATCAATCCTGAAGTGAAAGATTTTTATGAC
TTTACCATTGATGATTTCAAGTTAATCAACTATAAACATGGGGACA
AGCTTTTATTTGAGGTAGCGGTTTAATGCTGCCTTTTTATTGTGCA
GTGAATAGATAGCAGGTATCCTAATTTCATTAAGCAATCTGGAAGA
TGAATAAAAATTGAAGGACAAACACGTATAATACATAAAAAAGATT
AACTCTACAGTTAATCTTTTTTATTCAGAAGAAAATATCCTAACTT
TGAAACTAAATACAAAGTAAAAGCAATCATTACAGTTCTAGATATT
ACAATTCCATGAATAGCTAGATCATATCCAGCAGGTATCAACGCAT
TTGTATTACACATAAAATATATAGATATTAGAAGTGCTACAATAAC
TAAAATCATTCCAAAAAGACTTGTTTTTTCATATTTCATACCAATT
TCCACCCTTATTAAAGTTAGGTTTAAACAAAAGAGCTGAAGAAACG
AACTATGACCAGTATGCTCCAAGGAAAACCGCCAGACAATGCTGGC
GGCTTTTTGCTGCTTCGTTTATTTATTAACAGAGATCGTAACGTTA
TTTCCTGCAACTGAAACCTTTGCGAAATCC

The bold sequence represents the thyA coding sequence, the non-bold sequence represents the downstream region. Preferably, therefore, the vector comprises a nucleic acid sequence substantially as set out in SEQ ID NO: 4 or a fragment or variant thereof.

Any suitable multiple cloning site could be used in a vector of the invention. For instance, in an embodiment, the multiple cloning site may be encoded by the nucleic acid sequence provided herein as SEQ ID NO: 5 as follows:

[SEQ ID NO: 5]
AGCTTGCATGCCTGCAGGTCGACTCTAGAGGATCCCCCGGGGGTAC
CGAGCTCGAATTC

Preferably, therefore, the vector comprises a nucleic acid sequence substantially as set out in SEQ ID NO: 5 or a fragment or variant thereof.

In one purely illustrative embodiment, the thyA vector may comprise a nucleic acid sequence which may be encoded by the nucleic acid sequence provided herein as SEQ ID NO: 6 as follows:

[SEQ ID NO: 6]
CAGATCATATAAGGAATGAACCGCTGCCAAATATCATAAAAAAGTT
GTTAATGATCAAATGAATGAAATTAGAGAGAATTTATTTTAAAGAA
AGCCCAATTGCACATGGACAAATGACAATTGATATTGAGGTAAAAT
ACAATGCATTAAATCAGAAAAAGCTATTGGAGGATTTAATGTGTTT
AGACAATTTCCAATTTGGTATACACAAACACCTGACTATTTGAATT
TTTATGTACCGCAATATCAAACCATTTCGTATAATCCTCAACAATG
TTATCAACGGTGTATGTACCAAACTGGCGGTAACTATGAGCTATGT
GACAGACTATGTTATGGAGAAATACAGGTGTAAAAGAGGGGGATTA
ACTCCTCTTTAAACACACAGTGAGTGGAATAAGATCCTCACTTTAT
CTGCAAGTGCTTAGTATTTGCGATAATATTGCATTCGTAATAAATT
ATGCTTAGCAACTGAAAATGAAAGAAGGATATGAATAGTCATGACG
CAATTCGATAAACAATACAATTCAATTATAAAGGATATTATCAATA
ATGGAATCTCAGACGAAGAGTTTGATGTAAGAACCAAGTGGGACTC
AGATGGAACACCGGCACATACTCTAAGTGTAATCAGTAAGCAAATG
AGATTCGACAACTCAGAGGTTCCGATTTTAACGACAAAAAAGGTTG
CCTGGAAAACAGCCATTAAAGAGTTGCTCTGGATTTGGCAGCTGAA
ATCTAATGATGTTAATGATTTAAACATGATGGGCGTCCATATTTGG
GATCAGTGGAAACAAGAAGACGGAACCATCGGACATGCATATGGAT
TTCAGCTGGGGAAGAAAAACAGAAGTCTAAATGGAGAAAAAGTGGA
TCAGGTAGACTATCTTCTTCATCAATAAGCTTGCATGCCTGCAGGT
CGACTCTAGAGGATCCCCCGGGGGTACCGAGCTCGAATTCTGAAGA
ACAATCCATCTTCACGCAGACACATTACAATGCTGTGGAATCCTGA
TGAATTAGACGCAATGGCCTTAACGCCATGTGTATACGAGACACAA
TGGTACGTTAAACATGGGAAACTCCACCTTGAGGTAAGAGCACGGA
GCAATGATATGGCATTGGGAAATCCATTCAATGTATTCCAGTATAA
TGTGTTGCAGCGCATGATTGCTCAAGTGACTGGTTATGAGCTTGGT
GAATATATCTTTAACATTGGGGATTGCCATGTGTACACACGTCATA
TAGACAATTTGAAAATTCAAATGGAAAGAGAACAGTTTGAAGCACC
TGAACTATGGATCAATCCTGAAGTGAAAGATTTTTATGACTTTACC
ATTGATGATTTCAAGTTAATCAACTATAAACATGGGGACAAGCTTT
TATTTGAGGTAGCGGTTTAATGCTGCCTTTTTATTGTGCAGTGAAT
AGATAGCAGGTATCCTAATTTCATTAAGCAATCTGGAAGATGAATA
AAAATTGAAGGACAAACACGTATAATACATAAAAAAGATTAACTCT
ACAGTTAATCTTTTTTATTCAGAAGAAAATATCCTAACTTTGAAAC
TAAATACAAAGTAAAAGCAATCATTACAGTTCTAGATATTACAATT
CCATGAATAGCTAGATCATATCCAGCAGGTATCAACGCATTTGTAT
TACACATAAAATATATAGATATTAGAAGTGCTACAATAACTAAAAT
CATTCCAAAAAGACTTGTTTTTTCATATTTCATACCAATTTCCACC
CTTATTAAAGTTAGGTTTAAACAAAAGAGCTGAAGAAACGAACTAT
GACCAGTATGCTCCAAGGAAAACCGCCAGACAATGCTGGCGGCTTT
TTGCTGCTTCGTTTATTTATTAACAGAGATCGTAACGTTATTTCCT
GCAACTGAAACCTTTGCGAAATCC

Preferably, therefore, the vector comprises a nucleic acid sequence substantially as set out in SEQ ID NO: 6, or a fragment or variant thereof. Example primer annealing sites, which could be used to verify insertion of the heterologous gene, are shown in bold and underlined.

According to a fifth aspect of the invention, there is provided a vector for use in introducing a heterologous gene into the thyB gene within a spore-forming bacterium.

In an embodiment, the vector may comprise at least a portion of the 5′ portion of a Bacillus Spp. thyB gene, a heterologous gene, and at least a portion of the 3′ portion of a Bacillus Spp. thyB gene.

In an embodiment, the vector may comprise the left and right arms of a Bacillus Spp. thyB gene. The arms may flank a multiple cloning site. For instance, the vector may include the left (900 bp) and right (1.1 kb) arms of the B. subtilis thyB gene.

The length of the flanking arms may be adjusted for the length of the heterologous sequence to be inserted. Longer flanking arms (e.g. ˜1500 bp) may be necessary for large inserts.

The vectors may include a heterologous gene inserted at the multiple cloning site. The heterologous gene may be a chimeric gene comprising a spore coat protein fused to a heterologous gene. For instance, the spore coat protein may be CotB or CotC of B. subtilis. The vectors may comprise: the left arm of a Bacillus Spp. thyB gene, a spore coat protein, a multiple cloning site allowing insertion of a heterologous gene such that it is fused to the spore coat protein, and the right arm of a Bacillus Spp. thyB gene.

In a purely illustrative embodiment, the left arm of thyB may be encoded by the nucleic acid sequence provided herein as SEQ ID NO: 7, as follows:

[SEQ ID NO: 7]
CCAAATCTGCCGCTCAGTGTTTGCATGGAGAATGTAGAAAAAGTCC
TGAACAAACGTGAAATTATTCATGCTGTTTTGACAGGCCTTGCACT
CGATCAGCTTGCAGAACAGAAACTTCTCCCCGAACCGCTGCAGCAC
CTTGTTGAAACGGATGAACCGCTTTACGGCATAGATGAAATTATCC
CGCTTTCAATCGTTAATGTGTACGGGTCGATCGGTTTGACCAATTT
CGGTTATTTGGATAAAGAGAAGATTGGAATTATTAAGGAACTTGAT
GAAAGTCCAGACGGTATTCACACCTTTTTGGATGATATTGTGGCAG
CTCTTGCTGCAGCAGCGGCGAGCAGAATTGCACATACGCATCAGGA
TCTGCAAGATGAAGAAAAAGAACAGGATGAAAAGCCTGTCGTCAGC
TGACTATAAAAAAATCATTTCTGGGTTCAGAAATGATTTTTTATTG
TGTTACACTACTAGAAGACTACTTTTAAAGGATGAAAAAAATGAAA
CAGTATAAGGATTTCTGCAGACATGTTTTAGAGCATGGTGAGAAAA
AGGGAGACCGGACTGGGACCGGAACAATCAGCACTTTCGGATATCA
AATGAGATTTAATTTACGGGAAGGCTTTCCGATGCTCACCACTAAA
AAACTCCACTTTAAATCAATTGCGCATGAACTGCTGTGGTTCTTAA
AAGGAGATACGAATGTACGCTATCTGCAGGAAAACGGAGTGCGAAT
CTGGAATGAGTGGGCTGATGAAAACGGTGAACTTGGACCTGTATAT
GGCTCCCAATGGCGTTCTTGGCGGGGAGCTGATGGAGAAACCATTG
ATCAAATTTCCCGTCTTATTGAAGATATTAAAACAAATCCGAACTC
CAGACGCTTAATCGTCAGCGCCTGGA

The bold sequence represents the thyB coding sequence, the non-bold sequence represents the upstream region. Preferably, therefore, the vector comprises a nucleic acid sequence substantially as set out in SEQ ID NO: 7, or a fragment or variant thereof.

In one purely illustrative embodiment, the right arm of thyB may be encoded by the nucleic acid sequence provided herein as SEQ ID NO: 8, as follows:

[SEQ ID NO: 8]
ATGTTGGTGAAATTGATAAAATGGCGTTGCCGCCGTGCCATTGCCT
GTTCCAATTCTATGTGTCTGACGGCAAGCTGTCCTGTCAGCTGTAT
CAGCGCTCTGCCGATGTTTTCTTAGGTGTGCCGTTTAATATTGCAT
CTTATGCCCTCCTAACCATGATCATTGCTCATGTGACTGGGCTTGA
ACCGGGCGAGTTCATCCATACGTTTGGTGATGTTCATATTTACCAA
AATCATATTGAACAAGTCAATTTGCAGCTGGAAAGAGATGTTAGAC
CGCTTCCGCAGCTTCGTTTCGCCAGAAAGGTTGATTCTATTTTTAA
CTTTGCATTTGAGGACTTTATCATCGAGGATTATGATCCGCATCCT
CATATAAAAGGGGCGGTCAGCGTATGATTTCATTCATTTTTGCGAT
GGATGCCAACAGGCTTATCGGCAAAGACAATGATTTGCCGTGGCAT
TTGCCCAATGATCTTGCATACTTTAAGAAAATAACATCGGGCCATT
CAATCATTATGGGCCGGAAAACATTTGAATCGATCGGACGTCCGCT
TCCAAATCGGAAAAATATTGTCGTTACCTCAGCGCCGGATTCAGAA
TTTCAGGGATGCACGGTTGTCAGTTCATTAAAGGATGTACTGGACA
TTTGTTCAGGCCCTGAAGAATGCTTTGTGATCGGAGGGGCTCAGCT
CTATACGGACCTGTTCCCTTATGCGGACAGACTGTATATGACGAAA
ATTCATCACGAGTTTGAGGGTGACCGTCACTTTCCTGAATTTGATG
AATCCAATTGGAAGCTGGTTTCTTCTGAGCAGGGGACCAAAGACGA
AAAAAACCCGTATGATTACGAATTTCTAATGTATGAAAAAAAGAAA
TCTTCTAAAGCGGGAGGATTTTAATTGGTTCGCTACAGCCTTCTAG
TGGTTTATATTGTGTATATGCTGTTAAAAAATATGAAACAATTATT
TAATCAAACAATGCTCGATCCCCGTCTGTCATACAAAAAACAGATG
GCTCTTGTGTACGAACAGCCAAAGGCGTTTTTAGAAGGCTGTATCG
GCATCTCCGGTTCAGTTGTGACGATCCATCAGCCAGA

The bold sequence represents the thyB coding sequence, the non-bold sequence represents the downstream region. Preferably, therefore, the vector comprises a nucleic acid sequence substantially as set out in SEQ ID NO: 8, or a fragment or variant thereof.

The above illustrative embodiments of both thyA and thyB sequences are intended as an example. As the skilled person would be aware, the sequences will vary between species, in particular there is a great deal of variability between the upstream and downstream sequences.

Any suitable multiple cloning site could be used in a vector of the invention. For instance, in an embodiment, the multiple cloning site may be encoded by the nucleic acid sequence provided herein as SEQ ID NO: 9, as follows:

[SEQ ID NO: 9]
AAGCTTGCATGCCTGCAGGTCGACTCTAGAGGATCCCCCGGGGGTA
CCGAGCTCGAATTCTG

Preferably, therefore, the vector comprises a nucleic acid sequence substantially as set out in SEQ ID NO: 9, or a fragment or variant thereof.

In one embodiment, the thyB vector may comprise a nucleic acid sequence which may be encoded by the nucleic acid sequence provided herein as SEQ ID NO: 10, as follows:

[SEQ ID NO: 10]
CCAAATCTGCCGCTCAGTGTTTGCATGGAGAATGTAGAAAAAGTCC
TGAACAAACGTGAAATTATTCATGCTGTTTTGACAGGCCTTGCACT
CGATCAGCTTGCAGAACAGAAACTTCTCCCCGAACCGCTGCAGCAC
CTTGTTGAAACGGATGAACCGCTTTACGGCATAGATGAAATTATCC
CGCTTTCAATCGTTAATGTGTACGGGTCGATCGGTTTGACCAATTT
CGGTTATTTGGATAAAGAGAAGATTGGAATTATTAAGGAACTTGAT
GAAAGTCCAGACGGTATTCACACCTTTTTGGATGATATTGTGGCAG
CTCTTGCTGCAGCAGCGGCGAGCAGAATTGCACATACGCATCAGGA
TCTGCAAGATGAAGAAAAAGAACAGGATGAAAAGCCTGTCGTCAGC
TGACTATAAAAAAATCATTTCTGGGTTCAGAAATGATTTTTTATTG
TGTTACACTACTAGAAGACTACTTTTAAAGGATGAAAAAAATGAAA
CAGTATAAGGATTTCTGCAGACATGTTTTAGAGCATGGTGAGAAAA
AGGGAGACCGGACTGGGACCGGAACAATCAGCACTTTCGGATATCA
AATGAGATTTAATTTACGGGAAGGCTTTCCGATGCTCACCACTAAA
AAACTCCACTTTAAATCAATTGCGCATGAACTGCTGTGGTTCTTAA
AAGGAGATACGAATGTACGCTATCTGCAGGAAAACGGAGTGCGAAT
CTGGAATGAGTGGGCTGATGAAAACGGTGAACTTGGACCTGTATAT
GGCTCCCAATGGCGTTCTTGGCGGGGAGCTGATGGAGAAACCATTG
ATCAAATTTCCCGTCTTATTGAAGATATTAAAACAAATCCGAACTC
CAGACGCTTAATCGTCAGCGCCTGGAAAGCTTGCATGCCTGCAGGT
CGACTCTAGAGGATCCCCCGGGGGTACCGAGCTCGAATTCTGATGT
TGGTGAAATTGATAAAATGGCGTTGCCGCCGTGCCATTGCCTGTTC
CAATTCTATGTGTCTGACGGCAAGCTGTCCTGTCAGCTGTATCAGC
GCTCTGCCGATGTTTTCTTAGGTGTGCCGTTTAATATTGCATCTTA
TGCCCTCCTAACCATGATCATTGCTCATGTGACTGGGCTTGAACCG
GGCGAGTTCATCCATACGTTTGGTGATGTTCATATTTACCAAAATC
ATATTGAACAAGTCAATTTGCAGCTGGAAAGAGATGTTAGACCGCT
TCCGCAGCTTCGTTTCGCCAGAAAGGTTGATTCTATTTTTAACTTT
GCATTTGAGGACTTTATCATCGAGGATTATGATCCGCATCCTCATA
TAAAAGGGGCGGTCAGCGTATGATTTCATTCATTTTTGCGATGGAT
GCCAACAGGCTTATCGGCAAAGACAATGATTTGCCGTGGCATTTGC
CCAATGATCTTGCATACTTTAAGAAAATAACATCGGGCCATTCAAT
CATTATGGGCCGGAAAACATTTGAATCGATCGGACGTCCGCTTCCA
AATCGGAAAAATATTGTCGTTACCTCAGCGCCGGATTCAGAATTTC
AGGGATGCACGGTTGTCAGTTCATTAAAGGATGTACTGGACATTTG
TTCAGGCCCTGAAGAATGCTTTGTGATCGGAGGGGCTCAGCTCTAT
ACGGACCTGTTCCCTTATGCGGACAGACTGTATATGACGAAAATTC
ATCACGAGTTTGAGGGTGACCGTCACTTTCCTGAATTTGATGAATC
CAATTGGAAGCTGGTTTCTTCTGAGCAGGGGACCAAAGACGAAAAA
AACCCGTATGATTACGAATTTCTAATGTATGAAAAAAAGAAATCTT
CTAAAGCGGGAGGATTTTAATTGGTTCGCTACAGCCTTCTAGTGGT
TTATATTGTGTATATGCTGTTAAAAAATATGAAACAATTATTTAAT
CAAACAATGCTCGATCCCCGTCTGTCATACAAAAAACAGATGGCTC
TTGTGTACGAACAGCCAAAGGCGTTTTTAGAAGGCTGTATCGGCAT
CTCCGGTTCAGTTGTGACGATCCATCAGCCAGA

Preferably, therefore, the vector comprises a nucleic acid sequence substantially as set out in SEQ ID NO: 10, or a fragment or variant thereof. Example primer annealing sites, which could be used to verify insertion of the heterologous gene, are shown in bold and underlined.

According to a sixth aspect of the invention, there is provided a kit comprising reagents for use with the processes of the invention.

In an embodiment, the kit may comprise any medium as disclosed herein, for instance medium comprising YE at a concentration lower than 2 mg/ml, or comprising no YE. The kit may comprise trimethoprim. The kit may comprise medium comprising trimethoprim, optionally two mediums one with a higher level of trimethoprim than the other. The kit may comprise CAA. The kit may comprise one or more amino acids, or any combination or concentration as disclosed herein. The kit may comprise instructions describing the use of the kit or describing any process as disclosed herein. The kit may comprise at least one container for performing a process as disclosed herein, for instance a petri dish.

In an embodiment, the kit may comprise one or more vectors of the invention. The kit may comprise a vector for use in introducing a heterologous gene into the thyA gene within a spore-forming bacterium and/or a vector for use in introducing a heterologous gene into the thyB gene within a spore-forming bacterium. The kit may comprise a nucleic acid sequence including a heterologous gene, such as a gene for a vaccine, for a conjugation protein, or an enzyme. The kit may comprise a nucleic acid sequence including a spore coat protein. The spore coat protein may be configured to be easily attachable to a heterologous gene, for instance by proximity to a multiple cloning site.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID Nos: 1-19.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g., functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridises to the sequences shown or their complements, under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown.

Due to the degeneracy of the genetic code, it is clear that any nucleic acid sequence described herein could be varied or changed without substantially affecting the sequence of the protein encoded thereby, to provide a functional variant thereof.

Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

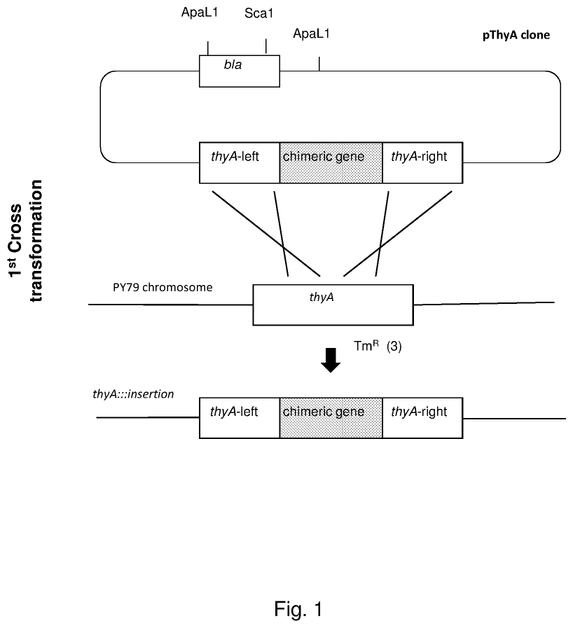

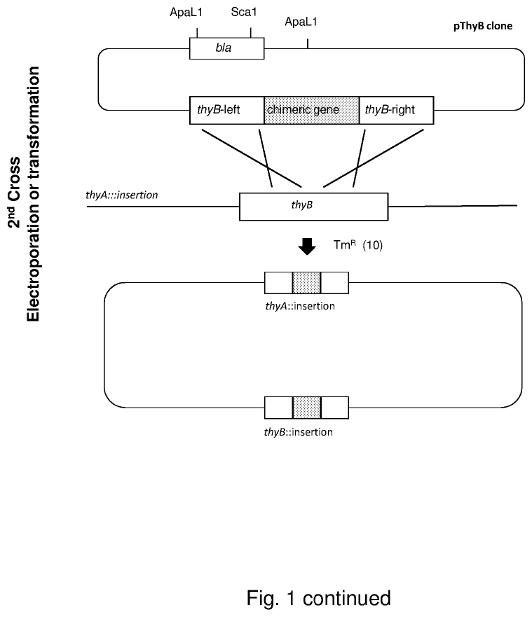

FIG. 1 is a graphical representation of the strain constructions. To construct ectopic insertions at the thyA and thyB loci of B. subtilis two steps are required. In step 1 a pThyA plasmid carrying a chimeric gene is linearised (ApaII digestion) and introduced into cells of a wild type B. subtilis strain (in this case strain PY79) by DNA-mediated transformation. Tm^{R} transformants are selected on SMM agar containing trimethoprim (3 μg/ml) and thymine (50 μg/ml) and carry an insertion of homologous thyA DNA together with the chimeric gene, by marker replacement, as shown. In the second step linearised plasmid DNA of a pThyB vector carrying the same or a different chimeric gene is introduced into cells of the thyA insertion strain created in the 1^{st} step. Selection for Tm^{R} is made on SMM+CAA agar containing trimethoprim (>6 μg/ml) and thymine (50 μg/ml);

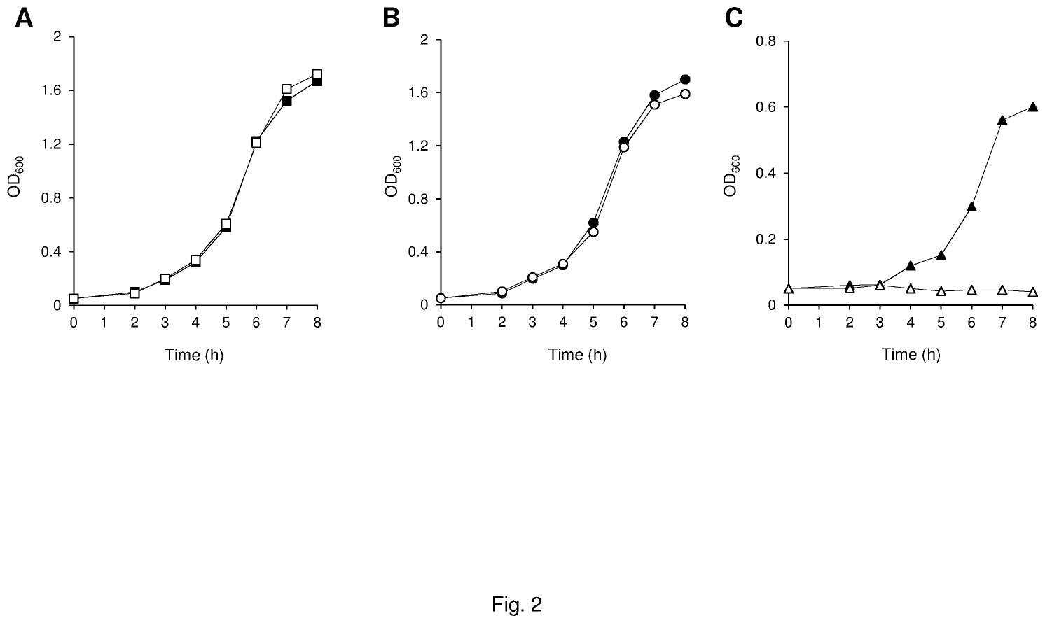

FIG. 2 is a graphical representation of growth of strains in minimal media. Wild type PY79 (panel A), SH13 thyA::cotB-tcdA₂₆₋₃₉ (panel B) and SH14 thyA::cotB-tcdA₂₆₋₃₉ thyB::cotC-tcdA₂₆₋₃₉ (panel C) were grown in SMM medium at 37° C. with (filled symbols) or without (open symbols) thymine supplementation (50 μg/ml);

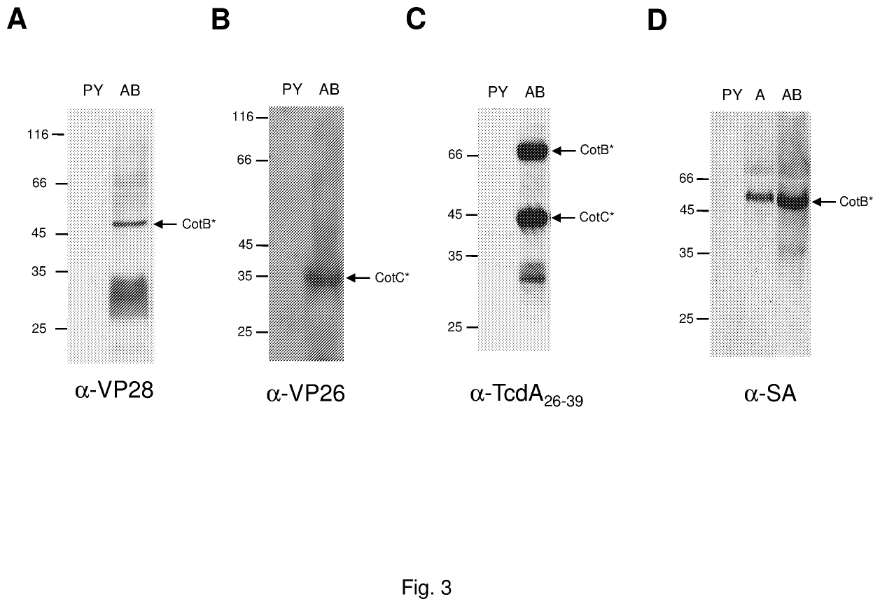

FIG. 3 shows spore coat expression. B. subtilis strains carrying insertions at the thyA and thyB loci were examined by Western blotting of spore coat proteins extracted from preparations of pure spores. Each panel shows bands obtained in extracts of wild type spores (PY79) or spores carrying thyA and thyB insertions (AB) or, in addition in panel D, spores carrying only a thyA insertion (A). Panels A and B show analysis of SH12 thyA::cotB-vp26 thyB::cotC-vp28 with anti-VP28 (panel A) and anti-VP26 (panel B) antibodies. Panel C shows analysis of SH14 thyA::cotB-tcdA₂₆₋₃₉ thyB::cotC-tcdA₂₆₋₃₉. Panel D, SH16 thyA::cotB-SA thyB::cotB-SA. Bands corresponding to the chimeric proteins are indicated (*) with the relevant coat protein anchor (CotB or CotC). The protein loaded per well corresponded to an extraction from 2×10⁸ spores (see Methods);

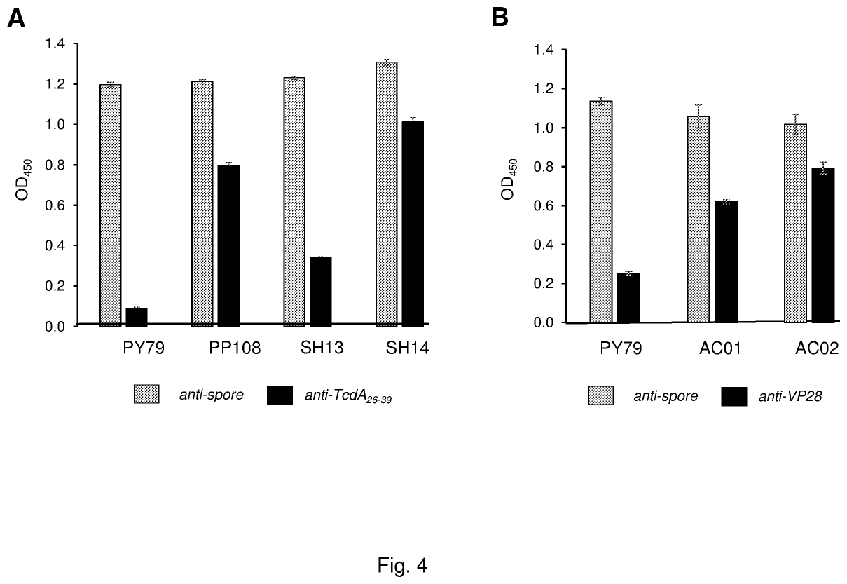

FIG. 4 is a graphical representation of surface expression determined by “Whole Spore ELISA”. Panel A; Microtiter plates were coated with spores (2×10⁸/well) of PY79 (spo⁺), PP108 (amyE::cotC-tcdA₂₆₋₃₉ thrC::cotB-tcdA₂₆₋₃₉), SH13 (thyA::cotB-tcdA₂₆₋₃₉) and SH14 (thyA::cotB-tcdA₂₆₋₃₉ thyB::cotC-tcdA₂₆₋₃₉) and then probed with either anti-spore (1:1,000) or anti-TdA₂₆₋₃₉ (1:500) rabbit PAbs. Secondary PAbs were 1:5,000 and naive serum was used for comparison, and basal levels were subtracted. Panel B; as for panel A but using spores of PY79, AC01(thyA::cotB-vp28) and AC02 (thyA::cotB-vp28 thyB::cotB-vp28) probed with either anti-spore (1:1,000) or anti-VP28 (1:300) rabbit PAbs;

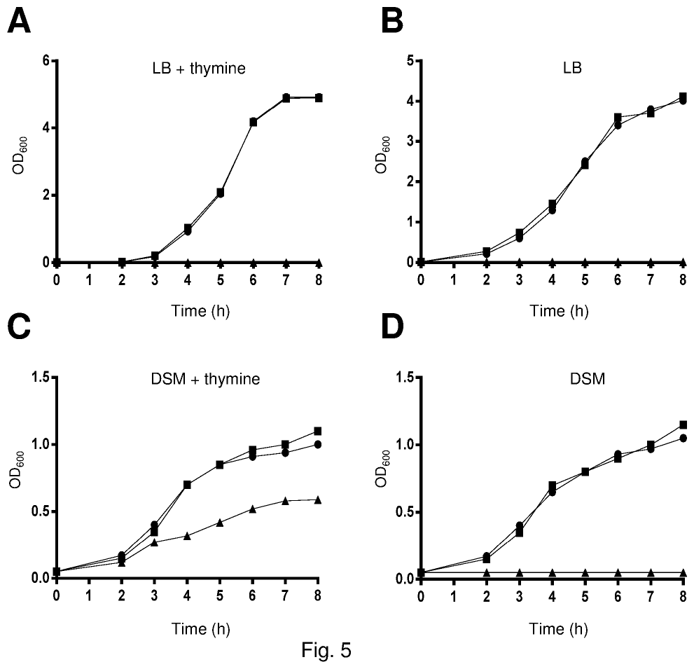

FIG. 5 is a graphical representation of growth of strains in rich media. Growth at 37° C. of PY79 (▪), SH13 (●) and SH14 (▴) were made in: panel A, LB+thymine (50 μg/ml); panel B, LB; panel C, DSM+thymine (50 μg/ml) and panel D, DSM;

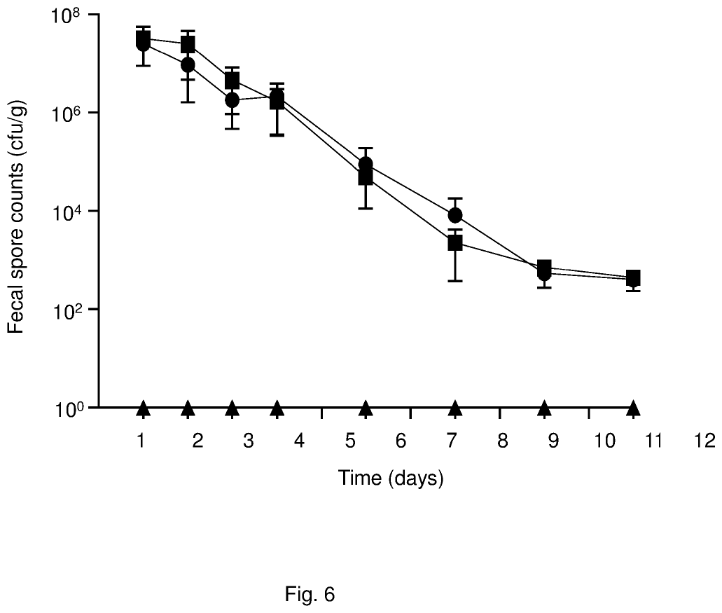

FIG. 6 is a graphical representation of survival in the murine GI-tract. Mice (n=5/gp) were given a single oral dose of spores (2×10⁹) of SH14 carrying insertions in the thyA and thyB loci or SH250, an essentially wild type strain tagged with a chloramphenicol resistance marker. The numbers of heat resistant spores in faeces was determined for 12 days post-dosing. SH14 numbers were determined on agar supplemented with trimethoprim and thymine (▪). In the absence of thymine no CFU was detectable (♦). SH250 CFU was determined on agar supplemented with chloramphenicol (●);

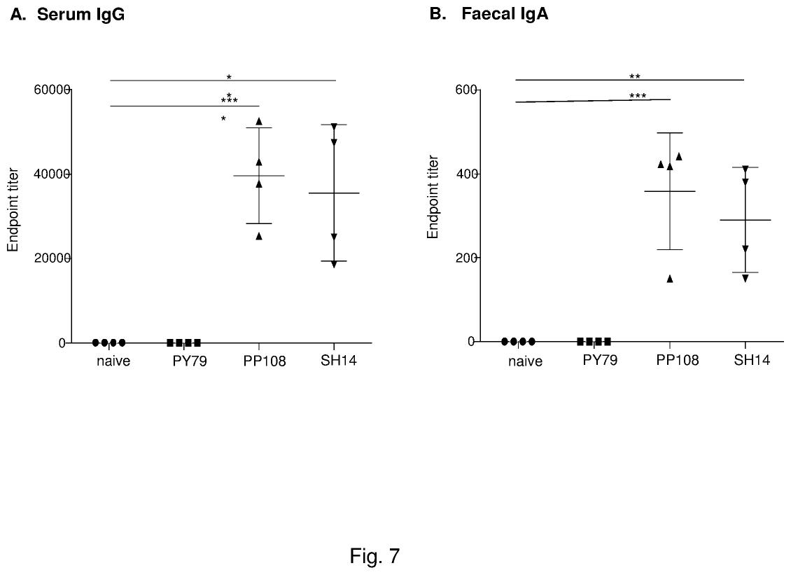

FIG. 7 is a graphical representation showing the immunogenicity of SH14 spores expressing the C. difficile TcdA₂₆₋₃₉ antigen. Spores (5×10¹⁰) of SH14 (thyA::cotB-tcdA₂₆₋₃₉ thyB::cotC-tcdA₂₆₋₃₉), PP108 (amyE::cotC-tdA₂₆₋₃₉ thrC::cotB-tcdA₂₆₋₃₉) and PY79 (spo⁺) were administered to mice (n=4) by oral gavage on days 1, 14, 35 and 57. Serum IgG (panel A) and fecal IgA (panel B) specific to TdA₂₆₋₃₉) was determined by ELISA and endpoint titers are shown. **, p<0.005; ***p<0.0002;

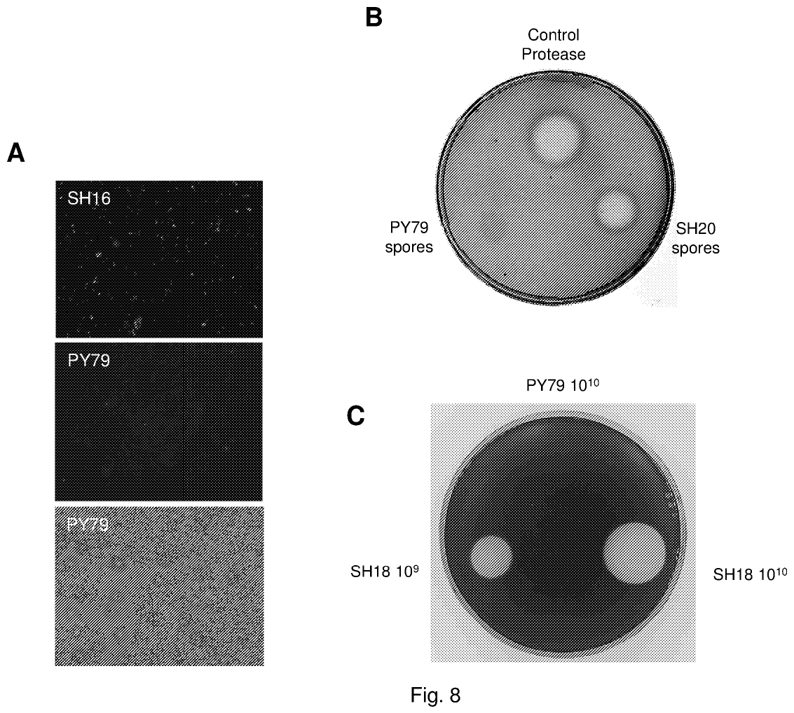

FIG. 8 is demonstrative of the conjugation of antibodies to spore-displayed streptavidin and enzyme display. Panel A shows visualisation of conjugation of anti-TcdA₂₆₋₃₉ antibodies to SH16 spores using immunofluorescence. As a control PY79 spores lacking SA failed to conjugate. The phase contrast image confirms the presence of PY79 spores. Panel B shows activity of subtilisin E displayed on the spore surface. Casein agar was used to visualise protease activity. Plates were spotted with 20 μl of suspensions of SH20 (thyA::aprE thyB::MCS) or PY79 spores. In each case the inoculum carried 5×10⁸ spores. As a positive control 0.02 units of Streptomyces griseus protease (Sigma P5147) was applied. After 24 h incubation at 37° C. plates were stained with Bromocresol green and incubated 30 min. at RT to reveal zones of degradation. Panel C shows starch plates stained with Lugol solution. Suspensions of pure spores of SH18 (10⁹ and 10¹⁰ cfu) or PY79 (10¹⁰ cfu) were spotted on plates and incubated for 48 h. Plates carried three antibiotics to prevent bacterial growth. Spores displaying active amylase produced a zone of clearing;

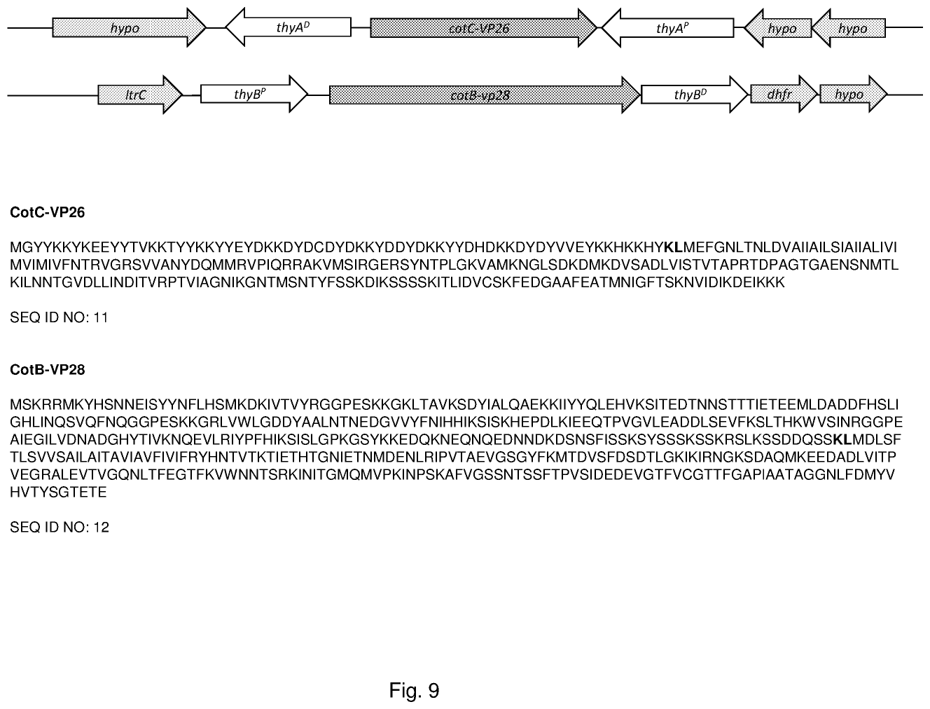

FIG. 9 shows schematic representations of thy insertions in SH12 (thyA::cotC-vp26 thyB::cotB-vp28). The proximal (^{P}) and distal (^{D}) segments of thyA and thyB flanking the chimeric gene insertion are shown. ORFS shown are: hypo, hypothetical coding ORFs, dhfr, dihydrofolate reductase, ltrC (Low temperature requirement C protein);

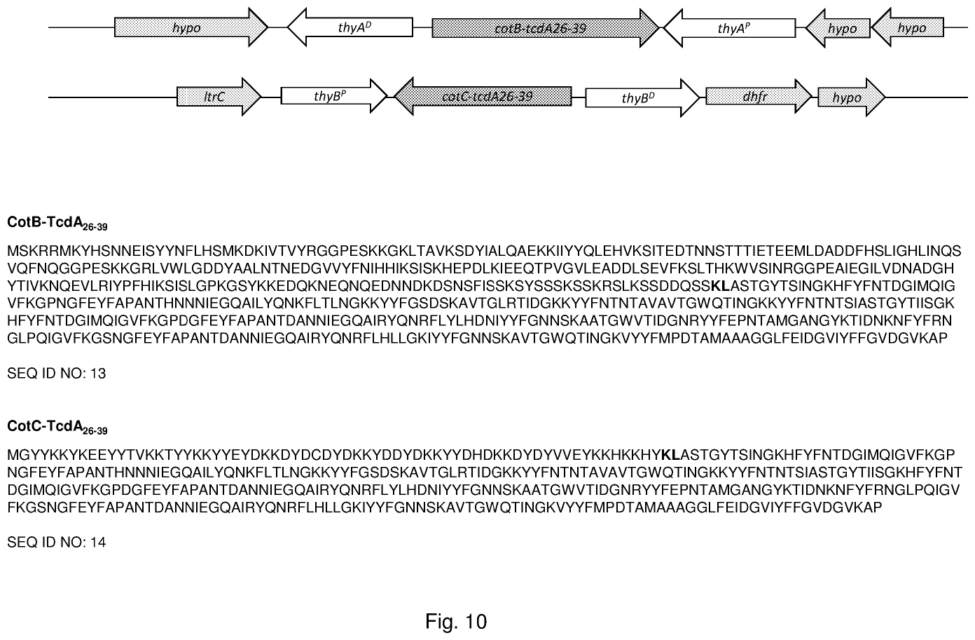

FIG. 10 shows schematic representations of thy insertions in SH14 (thyA::cotB-tcdA₂₆₋₃₉ thyB::cotC-tcdA₂₆₋₃₉). The proximal (^{P}) and distal (^{D}) segments of thyA and thyB flanking the chimeric gene insertion are shown. ORFS shown are: hypo, hypothetical coding ORFs, dhfr, dihydrofolate reductase, ltrC (low temperature requirement C protein);

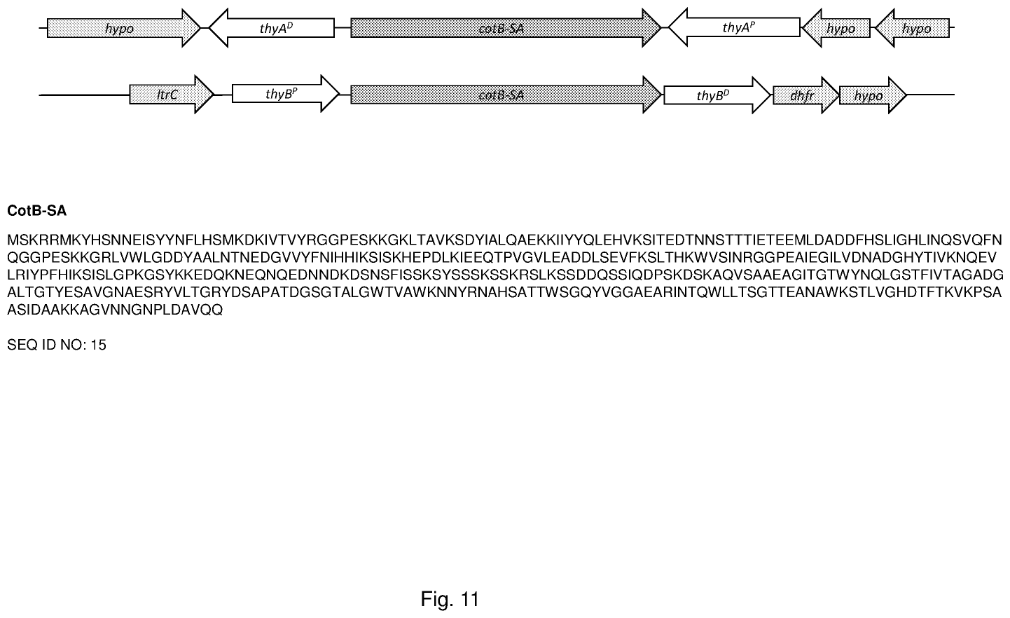

FIG. 11 shows schematic representations of thy insertions in SH16 (thyA::cotB-SA thyB::cotB-SA). The proximal (^{P}) and distal (^{D}) segments of thyA and thyB flanking the chimeric gene insertion are shown. ORFS shown are: hypo, hypothetical coding ORFs, dhfr, dihydrofolate reductase, ltrC (Low temperature requirement C protein);

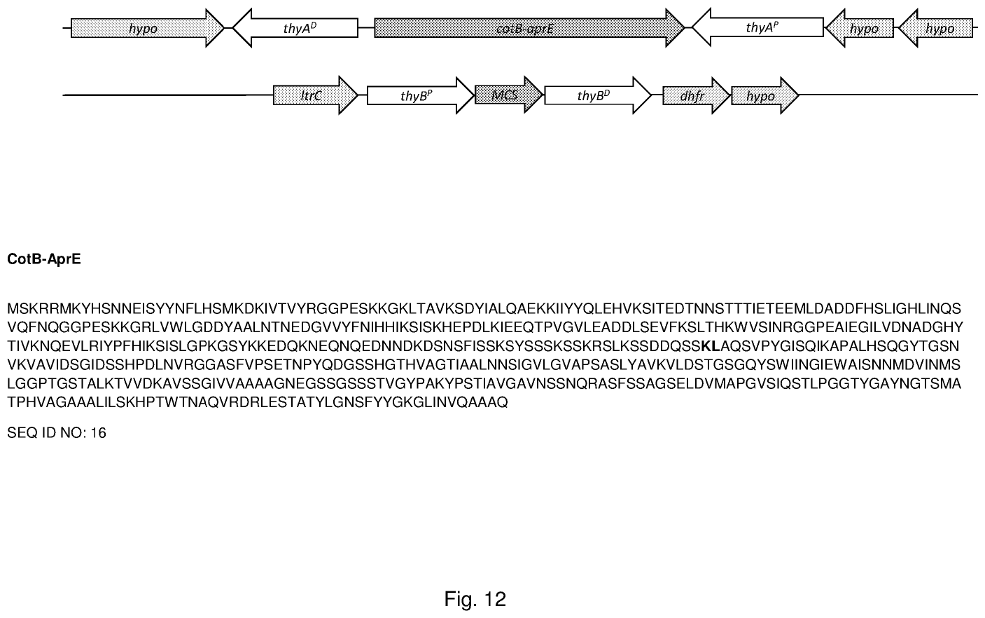

FIG. 12 shows schematic representations of thy insertions in SH20 (thyA::cotB-aprE thyB::MCS). The proximal (^{P}) and distal (^{D}) segments of thyA and thyB flanking the chimeric gene insertion are shown. ORFS shown are: hypo, hypothetical coding ORFs, dhfr, dihydrofolate reductase, ltrC (Low temperature requirement C protein);

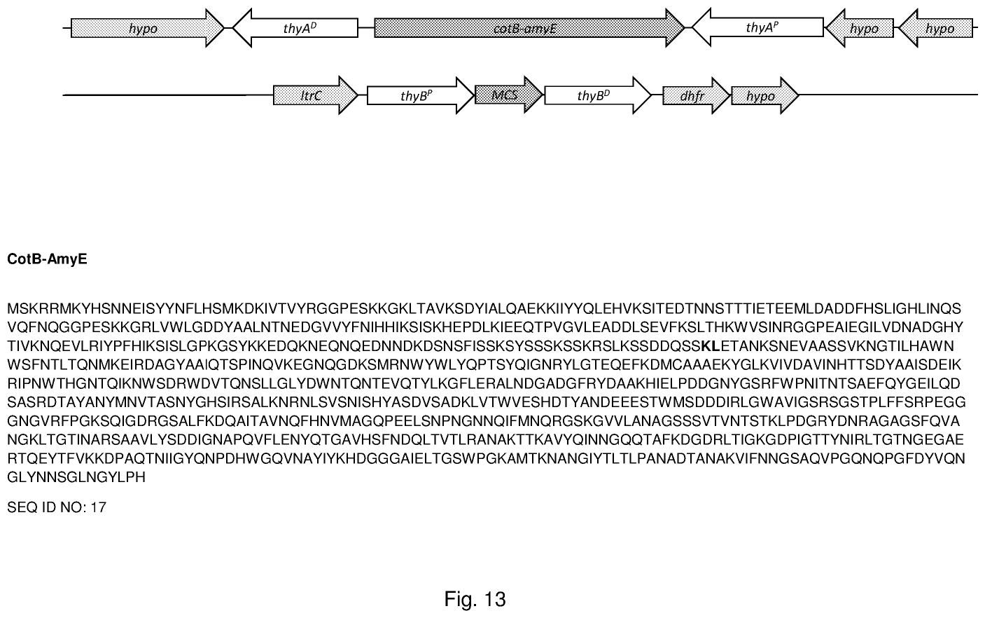

FIG. 13 shows schematic representations of thy insertions in SH18 (thyA::cotB-amyE thyB::MCS).The proximal (^{P}) and distal (^{D}) segments of thyA and thyB flanking the chimeric gene insertion are shown. ORFS shown are: hypo, hypothetical coding ORFs, dhfr, dihydrofolate reductase, ltrC (Low temperature requirement C protein);

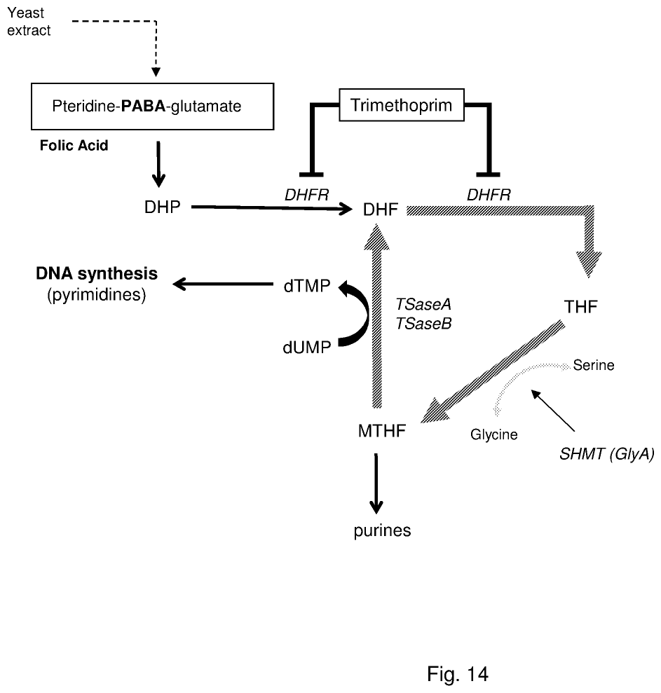

FIG. 14 is a schematic representation of the action of Trimethoprim on the Folate Pathway. Trimethoprim inhibits the enzyme dihydrofolic acid (DHFR) which is required for production of dihyrofolate (DHF) from dihydropterate (DHP) as well as for synthesis of tetrahydrofolic acid (THF). This prevents synthesis of methyleneterahydrofolic acid (MTHF) and synthesis of both purines and pyrimidines. In B. subtilis, two thymidylate synthetases (TS), TSase A and TSase B use MTHF to produce the pyrimidines thymidine and thymine. Inactivation of both TS enzymes is lethal unless exogenous thymine (or thymidine) is supplied. Also shown is para-aminobenzoic acid (PABA) which is a component of folic acid and SHMT (serine hydroxymethyltransferase);

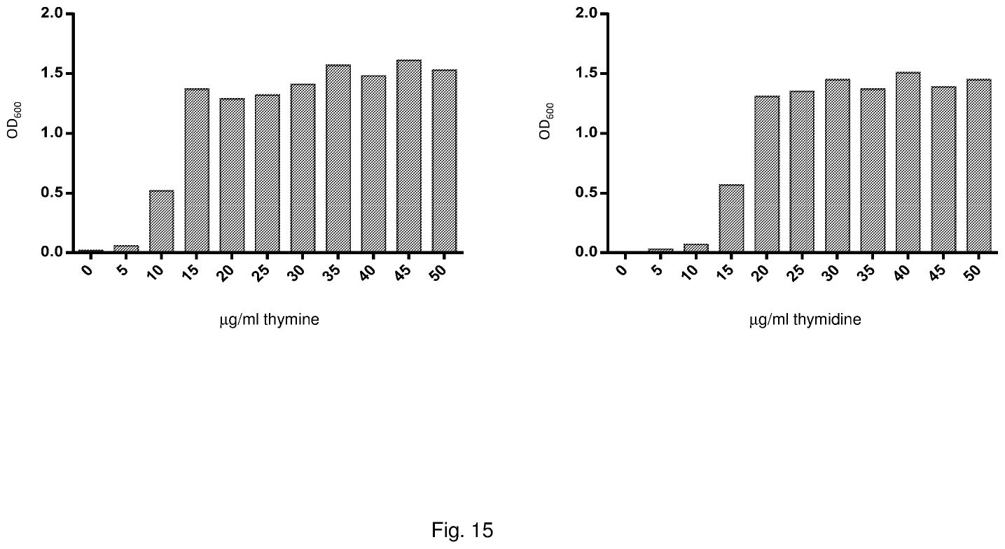

FIG. 15 shows a schematic representations of growth of SH14 (thyA::cotB-tcdA₂₆₋₃₉; thyB::cotC-tcdA₂₆₋₃₉) in medium containing thymine or thymidine. SH14 was grown in SMM containing 0.2% CAA with different concentrations of thymine or thymidine. Cultures incubated 18 h at 37° C. after which ODs determined;

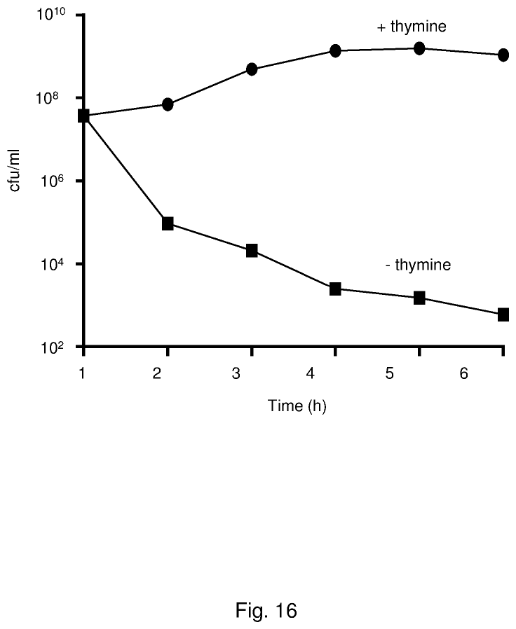

FIG. 16 shows schematic representations of loss of viability after removal of thymine. SH14 (thyA::cotB-tcdA₂₆₋₃₉thyB::cotC-tcdA₂₆₋₃₉) was grown (37° C.) in SOC medium containing trimethoprim (6 μg/ml) and thymine (50 μg/ml) overnight and then subcultured into fresh SOC supplemented with thymine (50 μg/ml). At an OD₆₀₀ of 0.5-0.6 the cells were harvested by centrifugation, washed 3× with PBS and suspended in 20 ml of warm SOC medium. 10 ml of this cell suspension was added to each of two flasks to which one thymine was added to give a final concentration of 50 μg/ml. The cultures were incubated at 37° C. with shaking in a water bath and viable cfu/ml determined at intervals by serial dilution and plating;

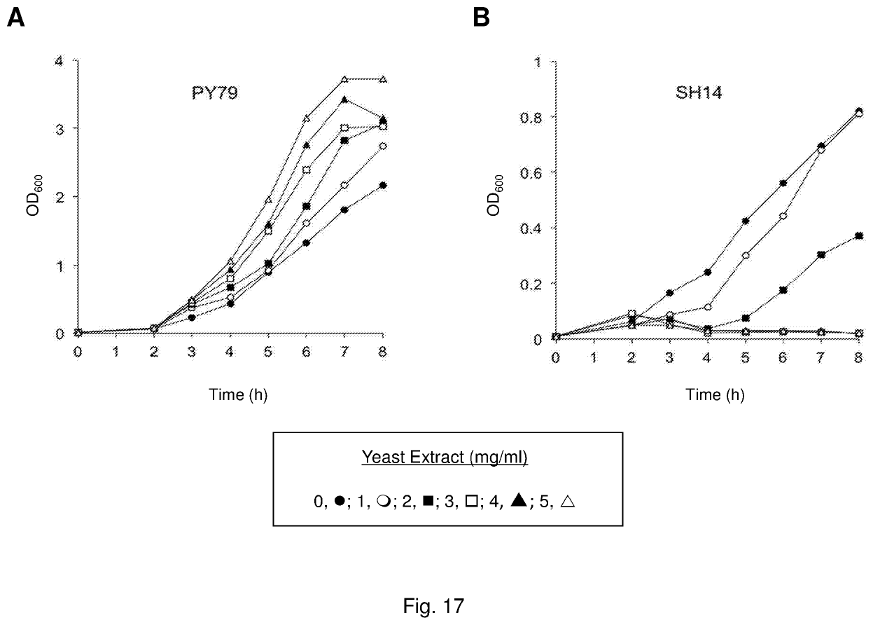

FIG. 17 shows schematic representations of growth of strains in rich media. Growth at 37° C. of PY79 (panel A), and SH14 (thyA::cotB-tcdA₂₆₋₃₉ thyB::cotC-tcdA₂₆₋₃₉) (panel B) were made in LB+thymine with different concentrations of yeast extract (mg/ml);

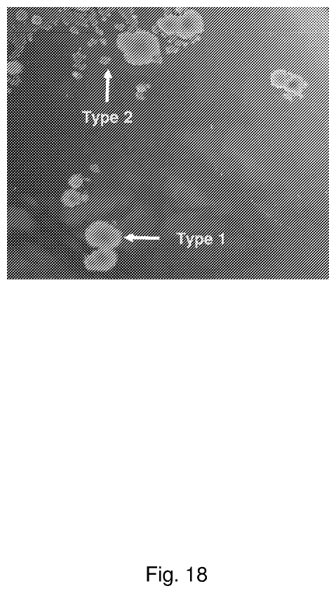

FIG. 18 shows colony types following the 1^{st} Genetic Cross. After transformation two type of colonies are seen: large-opaque (Type 1) and small-translucent (Type 2). Only the opaque colonies could be used for electroporation for construction of the double insertion strain; and

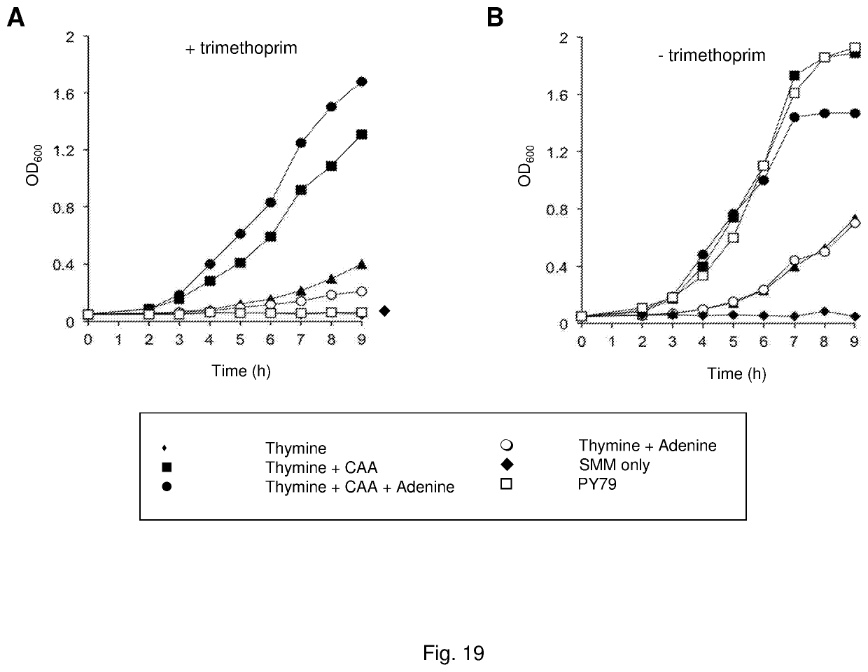

FIG. 19 shows schematic representations of growth of thyA thyB insertion strain in SMM media. Growth at 37° C. of SH14 (thyA::cotB-tcdA₂₆₋₃₉ thyB::cotC-tcdA₂₆₋₃₉) in SMM medium using different supplements as indicated (thymine 50 μg/ml, CAA (0.2% w/v,) Adenine (20 μg/ml). Media was supplemented with trimethoprim (3 μg/ml) in panel A and no trimethoprim in panel B. As a control B. subtilis strain PY79 was also evaluated for growth in SMM with no supplements.

### EXAMPLES

Genetic manipulation of bacterial spores of the genus Bacillus has shown potential for vaccination and for delivery of drugs or enzymes. Remarkably, proteins displayed on the spore surface retain activity and generally are not degraded. The heat stability of spores coupled with their desiccation resistance make them suitable for delivery to humans or to animals by the oral route. Despite these attributes one regulatory obstacle has remained regarding the fate of recombinant spores shed into the environment as viable spores. The inventors have addressed the biological containment of spore GMOs by utilizing the concept of a ‘thymine-less death’, a phenomenon first reported six decades ago but which has never been successfully put into practice for introducing heterologous genes into spore-forming bacteria. Using B. subtilis the inventors have inserted chimeric genes in the two thymidylate synthase genes, thyA and thyB using a two-step process. Insertion first at thyA followed by thyB where insertion generates resistance to trimethoprim enabling selection of recombinants. Importantly, this method requires introduction of no new antibiotic resistance genes. Recombinant bacteria or vegetative cells have a strict dependence on thymine or thymidine and in their absence cells lyse and die. Insertions are stable and no evidence for suppression or reversion was evident. Using this system the inventors have successfully created a number of spore vaccines as well as spores displaying active enzymes.

### Materials and Methods

### Strains, Media and General Methods

PY79 is a prototrophic strain of B. subtilis derived from the type strain 168 (28). PP108 (amyE::cotC-tcdA₂₆₋₃₉ thrC::cotB-tcdA₂₆₋₃₉) has been described elsewhere (4). SH250 is a prototrophic derivative of PY79 carrying the cat gene (encoding resistance to chloramphenicol) inserted at the amyE locus. General methods for work with B. subtilis including the ‘two-step transformation procedure’ were performed as described previously (29). DSM (Difco Sporulation Medium) is a standard medium for growth and sporulation of B. subtilis (30). For western blotting purified spores were prepared and 2×10⁸ suspended in 40 μl Bolt LDS buffer (Life Tech.) and incubated at 95° C. for 10 min. The spore suspension was centrifuged (10 min. 18,000 g) and 20 μl of supernatant run on a 12% SDS-PAGE gel.

pThy Vectors

pThyA (4,274 bp) carried a 1,910 bp segment comprising the left (900 bp) and right (950 bp) arms of the B. subtilis thyA gene surrounding a multiple cloning site (MCS) cloned into pMA-RQ (2,556 bp; Genscript, USA). Both arms carried additional proximal and distal DNA sequences adjacent to thyA. Similarly, pThyB1(4,973 bp) carried a 2,057 bp segment comprising the left (900 bp) and right (1.1 kb) arms of the B. subtilis thyB gene surrounding a MCS cloned into pBluescript SK(+) (2,958 bp). Plasmids carried the bla gene and the nucleotide sequences of the thyA and thyB segments are given in SEQ ID NO: 6 and SEQ ID NO: 10 (respectively) and shown schematically in FIG. 1. pThyA and pThyB plasmids were constructed which carried chimeric genes inserted at the MCS sites of each vector. Chimeric genes (not optimised for codon usage) containing an in-frame fusion between the 5′ segment of B. subtilis cotB or cotC to the vp26, vp28, tcdA₂₆₋₃₉ or SA coding ORFs were first synthesised with suitable 5′ and 3′ ends for sub-cloning in the MCS of the pThyA and pThyB vectors. For two genes, aprE and amyE the inventors amplified from chromosomal DNA templates and then fused to cotB. The aprE gene (encoding subtilisin E) was PCR amplified from a B. subtilis strain (SG115) in our collection and the coding segment amplified lacked the N-terminal regions involved in protein secretion (pre) and activation (pro) (31). The amyE gene (encoding alpha amylase) was amplified from a lab strain of Bacillus amyloliquefaciens and was cloned devoid of the secretory signal sequence. The gene fusions used for this work are shown in FIGS. 9 to 13.

Construction of B. subtilis strains carrying thyA and thyB insertions The procedure developed here consisted of two steps. In the first stage cells of a wild-type recipient strain (in the work described here the prototrophic strain PY79 was used) were made competent using a ‘two-step transformation’ procedure described by Dubnau and Davidoff-Abelson (33) and in common use in Bacillus labs (33, 34). pThyA plasmids carrying the chimeric gene were linearised with either ApaLI or ScaI digestion and cells plated on SMM (Spizizen's Minimal Medium (SMM) (29) agar supplemented with thymine (50 μg/ml) and trimethoprim (3 μg/ml). After 72 h-96 h of growth single colonies were colony purified and assessed for growth at 37° C. and 46° C. on SMM agar±thymine (50 μg/ml)+trimethoprim (3 μg/ml). Cells carrying an insertion at the thyA locus would grow at 37° C. with or without thymine but were unable to grow at 46° C. unless supplemented with thymine. A further verification was to amplify, by PCR, the presence of the chimeric gene from transformants using primers annealing to the thyA sequences. Typically transformation frequencies were about 2×10³/μg of competent cells with about 15-20% of colonies carrying the correct insertion (see later for explanation). In the second stage, a linearised (ApaL1 or Sca1) pThyB plasmid carrying a chimeric gene was introduced into cells of the thyA insertion strain by electroporation. Electroporated cells were plated on SMM+CAA (SMM containing 0.2% (w/v) casamino acids (CAA)) containing thymine (50 μg/ml) and trimethoprim (6 μg/ml) and incubated at 37° C. for 48 h. To confirm the presence of both a thyA and thyB insertion colonies were streaked on SMM+CAA agar±thymine (50 μg/ml) and grown at 37° C. Cells carrying two insertions were unable to grow at both 37° C. and 46° C. unless supplemented with thymine. A final verification was made using PCR primers that amplified the two insertions. Using electroporation, integration frequencies were about 1×10³/μg of linear DNA with ˜20% of trimethoprim resistance colonies carrying two insertions (thyA and thyB).

### Electroporation

The procedure used here was modified from established methods (35) for electroporation in Bacillus primarily with the use of SOC2 medium that contained no yeast extract. SOC2 was tryptone (2% w/v), NaCl (10 mM), KCl (2.5 mM), MgCl₂ (5 mM), MgSO₄.7H₂O (5 mM) and glucose (20 mM). An overnight culture of the strain carrying a thyA insertion was sub-cultured in 25 ml of SOC2 medium (supplemented with 0.5M sorbitol) to give a starting OD₆₀₀ of 0.2. The culture was grown at 37° C. to an OD₆₀₀ of 1.4, cooled on ice for 10 min. and then harvested by centrifugation at 4° C. (5,000×g, 5 min.). Cells were washed 4-times in ice-cold electroporation solution (0.5M sorbitol, 0.5M mannitol, 10% (v/v) glycerol) and suspended in 1.6 ml of the same ice-cold solution. The cells were now electro-competent and ready for immediate use. Cells were kept on ice and used within 30 min. although aliquots could be stored at −80° C. 1 μl (˜50 ng) of linearised plasmid DNA was added to 60 μl of electro-competent cells and the mixture transferred to a pre-chilled cuvette (1 mm gap width) and incubated for 1.5 min. on ice. The cuvette was then placed inside the electroporator (BioRad GenePulser Xcell) and the following parameters used for electroporation: Voltage 2, 100V, resistance 200 W, time, 5 milliseconds and number of pulses, 1. After electroporation 1 ml of recovery medium (SOC2 medium containing 0.5M sorbitol and 0.38M mannitol) was added to the cuvette and the mixture transferred to a 2 ml Eppendorf and incubated for 3 h in 37° C. after which cells were serially diluted and plated on appropriate selective media.

### Whole-Spore ELISA

An ELISA method was used to detect surface exposed proteins as described previously (36). Microplate wells were coated with 50 μl of a suspension of pure spores (2×10⁸ spores/well) which left overnight at 4° C. Plates were blocked with 2% (w/v) BSA for 1 h at 37° C. Rabbit polyclonal antibodies recognizing either the heterologous antigen expressed on the spore surface or the whole B. subtilis spore were used as primaries with incubation for 2 h at RT. Anti-rabbit IgG-horseradish peroxidase (HRP) conjugate (1:5,000 in PBS plus 0.05% Tween-20) was used as a secondary with 1 h incubation at RT. TMB (3, 3′, 5, 5′-tetramethylbenzidine) was used as substrate.

### Antibodies

Polyclonal antibodies to VP28 and TcdA₂₆₋₃₉ were raised in rabbits using four sub-cutaneous injections (1 mg/dose, every 14 days). Recombinant proteins were complexed with Freund's adjuvant and serum purified using protein A chromatography. VP26 polyclonals were raised in mice using purified rVP26 protein (4 intra-peritoneal doses at 14-day intervals, 10 μg/dose, with Freund's adjuvant).

### Animal Studies

For immunogenicity studies mice (C57 Black 6, female, age 7 weeks) were dosed with preparations of pure spores of PY79 or SH14. Immune responses to TcdA₂₆₋₃₉ in serum and faecal samples were made as described previously (4). For longevity studies Balb/c mice (female, age 7-8 weeks) were used. Mice (n=5) were administered a single dose of pure spores (2×10⁹) of SH14 or SH250 by oral gavage. At times thereafter freshly voided faeces was collected (3-4 pellets), homogenised and for SH14 faeces serial dilutions plated on (i) DSM and (iii) DSM+trimethoprim (6 μg/ml)+thymine (50 μg/ml) agar plates. SH250 faecal dilutions were plated on DSM plates containing chloramphenicol (5 μg/ml). Individual SH14 colonies were randomly checked for the presence of the thy insertions using PCR.

### Conjugation to Streptavidin

Polyclonal antibodies (rabbit; 100 μg) raised to rTcdA₂₆₋₃₉ protein were biotinylated using the Lightning-Link Rapid biotin conjugation kit type A (Innova Biosciences). Purified spores (1×10⁹) of strain PY79 or spores expressing CotB-SA (SH16) in 200 μl of PBS were mixed with 1 μg of biotinylated antibody and incubated overnight at 4° C. Spores were then washed four-times with PBS and suspended in 1 ml of PBS. ˜3×10⁸ of conjugated spores were used to coat microplate wells which were then probed with an anti-rabbit IgG-horseradish conjugate (1:5,000 in PBS plus 0.05% Tween-20) with 1 h incubation at RT followed by three washes before TMB (3,3′,5,5′-tetramethylbenzidine) colour development. As a control PY79 spores were taken through the same procedure. For immunoflourescence 5×10⁶ of treated spores of SH16 or PY79 were added to microscope slides and allowed to air dry. After three washes with PBS slides were blocked with PBS containing 2% (w/v) BSA plus 0.05% (v/v) Tween-20 for 45 min. at 37° C. Biotinylated anti-TdA₂₆₋₃₉ antibodies (1:300 diln.; 200 μl) were added to slides, incubated for 30 min. at RT and then washed 3-times with PBS+0.05% (v/v) Tween-20. Rabbit FITC serum (Sigma F0382 at 1:200 diln.) was added and slides incubated for 30 min. at RT. Image analysis was done with an EVOS fl LED microscope.

### Toxin Subtraction Assays

C. difficile strain RT176 (tcdA⁺ tcdB⁺) was grown in TY broth (3% (w/v) tryptose, 2% (w/v) yeast extract and 0.1% (w/v) sodium thiodlycolate) for 24 h at 37° C. The cell free supernatant was filter-sterilised and kept at 4° C. till assay. The minimum lethal concentration (MLC) of supernatant required to cause 100% toxicity to HT29 cells was determined using 2-fold dilution and addition of the diluted lysate to HT29 cells followed by assessment of toxicity using a cell rounding assay (4). For the assay 10⁹ pure spores of SH16 spores conjugated to TcdA₂₆₋₃₉ polyclonal antibodies (see above) were added to 200 μl of 2% McCoy's medium containing the MLC of toxins (typically a 1/4000 dilution). The mixture was incubated for 5 min. at RT and then cytotoxicity assessed using HT29 cells and incubation for 24 h. As a control PY79 spores that had been mixed with TcdA₂₆₋₃₉ antibodies (see above) were used in parallel.

### Enzyme Activity

Protease activity was determined using the Universal Protease Assay described by Sigma Aldrich on their web site using casein as a substrate. Casein agar was 1% (w/v) casein, 1% (w/v) skimmed milk, 1% (w/v) and 1.2% (w/v) agar technical No. 2, Oxoid). Amylase activity in liquid was measured as described (37). Production of active amylase was tested by applying suspensions of spores (volume of 20 μl) to agar plates carrying only soluble starch (1% w/v) and beef extract (0.3% w/v) and three antibiotics (trimethoprim 10 μg/ml, chloramphenicol 30 μg/ml and erythromycin 30 μg/ml). Antibiotics were used to prevent any bacterial growth on the plates ensuring activity arose from dormant spores only. Plates were incubated 48 h at 37° C. after which the plate was flooded with Lugol solution (Sigma) for 2 min. to reveal zones of starch degradation.

### Example 1

### Rationale

An absolute requirement for thymine in B. subtilis requires two thymidylate synthases (TSase) encoded by the unlinked thyA (TSaseA) and thyB (TSaseB) genes (27). These genes are linked to the folate pathway and provide pyrimidines for cell growth (FIG. 14). TSaseB is thermo-sensitive and retains only ˜5-8% activity at a restrictive temperature of 46° C. Thus, inactivation of the thyA locus requires supplementation with thymine (or thymidine) for growth at 46° C. TSaseA is not thermo-sensitive and so inactivation of thyB allows cells to grow at an elevated temperature. Inactivation of both thyA and thyB however, produces an absolute requirement on thymine for growth at both 37° C. and 46° C. As shown by Neuhard et al (27) inactivation of the thy genes produces resistance to the anti-folate drug trimethoprim (or aminopterin) since the need for dihydrofolate reductase (DHFR), the target for trimethoprim, is dispensed with. However, the level of resistance differs, with insertion at thyA producing a lower level of resistance than that found in a thyA thyB mutant (27). This attribute of differential resistance to trimethoprim enables a novel, two-step, ectopic cloning system to be designed (FIG. 1). In the first step, a gene is introduced at the thyA locus followed, in the second step, by insertion at thyB using resistance to increasing concentrations of trimethoprim for positive selection.

To demonstrate proof of concept for ectopic cloning at the thy loci the inventors chose a number of heterologous genes whose products had previously been expressed on the spore surface. In each case expression had been achieved by fusion to a B. subtilis gene encoding a surface expressed spore coat protein (either CotB or CotC). The proteins chosen were VP26 and VP28, both envelope proteins of the shrimp virus WSSV (White Spot Syndrome Virus). When displayed on B. subtilis spores and incorporated in feed these recombinant spores have been shown to confer protection to shrimps challenged with WSSV (7, 8, 9). (ii) TcdA₂₆₋₃₉ encoding a C-terminal domain of Clostridium difficile toxin A (TcdA) expressed on the spore surface has been shown to confer protection to C. difficile infection (CDI) in hamsters (4, 38) dosed orally with these recombinant spores. (iii) Streptavidin (SA) when expressed on spores can be conjugated to the monoclonal antibody Cetuximab enabling targeting to colon cancer cells (15). (iv) Finally, two enzymes, subtilisin E (AprE), an alkaline protease and alpha amylase (AmyE) which are both enzymes of industrial importance and commonly incorporated in animal feed (32, 39).

### A Two-Step Method for Ectopic Gene Insertion

Two plasmids, pThyA and pThyB, were designed and synthesised that carry the complete thyA or thyB genes interrupted midpoint with a multiple cloning site (MCS) for insertion of heterologous DNA (FIG. 1, SEQ ID NO: 6, and SEQ ID NO: 10). To ensure sufficient DNA homology to enable a double crossover recombination event, the left and right arms each carried the relevant thy segments as well as flanking upstream or downstream DNA (a total of ˜900 bp proximal and distal). In-frame fusions of the cotB and cotC genes (encoding the spore coat anchors required for display of heterologous proteins) to vp26, vp28, tcdA₂₆₋₃₉, SA, aprE or amyE ORFs (FIGS. 9 to 13) were then cloned into the appropriate pThyA or pThyB vectors. The pThyA plasmids were next linearised and used to transform competent cells of the wild type strain PY79 with selection for trimethoprim resistance on plates supplemented with trimethoprim (3 μg/ml) and thymine at 37° C. Transformants carrying thyA insertions could be recognised by their failure to grow at 46° C. without thymine. In the second step, for introduction of insertions at the thyB locus classical DNA-mediated transformation of competent cells with the pThyB plasmid proved inefficient with low integration frequencies. Instead, the inventors developed an electroporation method that reliably and reproducibly enabled introduction of pThyB plasmids at the thyB locus in thyA⁻ strains (see Methods) using selection with a higher concentration of trimethoprim (6 μg/ml). Strains carrying insertions at thyA and thyB were verified by their failure to grow at both 37° C. and 46° C. in the absence of thymine. Using this two-step process the inventors successfully constructed a number of strains carrying insertions (vp26, vp28, tcdA₂₆₋₃₉ and SA) at the thyA and thyB loci (Table 1). In addition, the inventors also made strains carrying single insertions of the aprE and amyE genes at the thyA locus. To create an absolute thymine dependence the inventors simply linearised an ‘empty’ pThyB plasmid and introduced this DNA into the thyA mutant selecting for trimethoprim at 6 μg/ml. Strains the inventors successfully constructed included monovalent (VP28, TcdA₂₆₋₃₉, streptavidin, subtilisin E and amylase) as well as divalent (expression of VP26 and VP28 on one spore) expression by fusion to one or two different spore coat anchors (CotB and CotC). For each strain constructed in Table 1 the inventors confirmed by nucleotide sequence analysis the integrity of the thyA or thyB insertion.

The inventors examined growth of the insertions in minimal SMM medium at 37° C. and FIG. 2 shows examples of a strain (SH14) carrying cotB-tcdA₂₆₋₃₉ and cotC-tcdA₂₆₋₃₉ insertions at the thyA and thyB loci. As expected the thyA insertion strain grew normally with or without thymine (FIG. 2B) and was indistinguishable from wild type PY79 (FIG. 2A). By contrast the thyA thyB insertion strain was thymine dependent but in the presence of thymine had reduced fitness as shown from the lower maximal OD (FIG. 2C.

In addition, using whole genome sequencing the inventors confirmed that strain SH12 (thyA::cotC-vp26 thyB::cotB-vp28) and SH14 (thyA::cotB-tcdA₂₆₋₃₉thyA::cotC-tcdA₂₆-39) carried the specific insertions at the thy loci without any chromosomal abberations (not shown). Expression of chimeric proteins was confirmed by Western blotting of proteins extracted from purified spores (FIG. 3 shows blots for detection of the VP26, VP28, TcdA₂₆₋₃₉ and SA chimeras in SH12, SH14 and SH16) demonstrating bands of the correct mwt. A second method was ELISA detection of whole spores using polyclonal antibodies to the corresponding heterologous antigen and FIG. 4 shows surface detection of the TcdA₂₆₋₃₉ (FIG. 4A) and VP28 (FIG. 4B) antigens by whole-spore ELISA. As expected, levels of the TcdA₂₆₋₃₉ protein carried at both the thyA and thyB loci (SH14 in FIG. 4A) was greater than expression at one locus (i.e., thyA, SH13). In parallel the inventors also measured the abundance of TcdA₂₆₋₃₉ in spores of PP108 that carried the same antigen fused to CotB and CotC but inserted at the thrC and amyE loci respectively (4). Expression levels were somewhat lower but it should be noted that using an anti-spore PAb to measure levels of spore coat proteins expression levels were correspondingly reduced.

Both thymine and thymidine could be used for growth of strains carrying insertions in the thyA and thyB loci. Using SH14 the inventors showed that for optimal growth 15 μg/ml of thymine or 20 μg/ml of thymidine was sufficient (FIG. 15). This was significantly less than the 50 μg/ml reported before (27) and most probably reflects differences in strain backgrounds used. Cell carrying insertions at the thyA and thyB loci although able to grow in media supplemented with thymine underwent a massive loss in cell viability (˜5 logs in 5 h) when thymine was removed, a hallmark of a ‘thymine-less death’ (FIG. 16).

TABLE 1
Phenotypes of B. subtilis recombinant strains

| | | 37° C.¹ | | 46° C.¹ | | Tm | Sporulation³ | | |
|---|---|---|---|---|---|---|---|---|---|
| Strain | Genotype | −thy | +thy | −thy | +thy | MIC² | TC | Spores | % |
| PY₇₉ | thyA⁺ thyB⁺ | + | + | + | + | 0.25 | 2.8 × 10⁸ | 2.4 × 10⁸ | 85.7 |
| SH₁₁ | thyA::cotC-vp26 | + | + | − | + | 16 | 1.9 × 10⁸ | 1.6 × 10⁸ | 82 |
| SH₁₂ | thyA::cotC-vp26 thyB::cotB-vp28 | − | + | − | + | >64 | 2 × 10⁸ | 1.5 × 10⁸ | 75 |
| AC₀₁ | thyA::cotB-vp28 | + | + | − | + | 16 | 2.6 × 10⁸ | 2.2 × 10⁸ | 85 |
| AC₀₂ | thyA::cotB-vp28 thyB::cotB-vp28 | − | + | − | + | >64 | 1.9 × 10⁸ | 1.5 × 10⁸ | 78.6 |
| SH₁₃ | thyA::COth-tedA₂₆₋₃₉ | + | + | − | + | 16 | 2.2 × 10⁸ | 1.8 × 10⁸ | 85 |
| SH₁₄ | thyA::cotB-tcdA₂₆₋₃₉ thyB::cotC-tcdA₂₆₋₃₉ | − | + | − | + | >64 | 2.5 × 10⁸ | 1.9 × 10⁸ | 77.2 |
| SH₁₅ | thyA::cotB-SA | + | + | − | + | 16 | 2.8 × 10⁸ | 2.3 × 10⁸ | 82 |
| SH₁₆ | thyA::cotB-SA thyB::cotB-SA | − | + | − | + | >64 | 3.1 × 10⁸ | 2.4 × 10⁸ | 77.4 |
| SH₁₇ | thyA::cotB-amyE | + | + | − | + | | | | |
| SH₁₈ | thyA::cotB-amyE thyB::MCS | − | + | − | + | | | | |
| SH₁₉ | thyA::cotB-aprE | + | + | − | + | 16 | 8.7 × 10⁸ | 5.5 × 10⁸ | 90 |
| SH₂₀ | thyA::cotB-aprE thyB::MCS | − | + | − | + | >64 | 8.1 × 10⁷ | 6.8 × 10⁷ | 83.9 |

¹growth (+) or no growth (−) on SMM agar with or without thymine (50 μg/ml)
²minimal inhibitory concentration (MIC; μg/ml) of trimethoprim (Tm) determined using a microdilution method (52).
³% of heat-resistant spores in cultures grown overnight in DSM medium (+thymine) at 37° C. TC, total count cfu/ml;
Spores, heat-resistant spore count cfu/ml.

Finally, using this cloning system the inventors were able to introduce insertions that were of the same or opposed transcriptional polarity to the respective thy gene (FIG. 9 to FIG. 13) showing no evidence of read through from the proximal thy coding sequences.

### Example 2

### Experimental Considerations for Use of Ectopic Insertion at the Thy Loci

### a) Growth in Rich Media

In rich medium such as LB and DSM the single thyA insertion strain was able to grow at 37° C. without thymine supplementation while the thyA thyB double insertion showed no growth however (FIGS. 5B and D). Intriguingly, supplementing LB or DSM with thymine did not restore normal levels of growth. In LB the double insertion failed to grow (FIG. 5A) while in DSM plus thymine limited growth was observed (FIG. 5C). However, after 24 h growth in DSM sporulation was shown to have occurred and the numbers of heat-resistant spores was essentially equivalent to PY79 (Table 1).

Since the thyA thyB insertion strain could grow, albeit with reduced fitness, in minimal medium supplemented with thymine (see FIG. 2C) the inventors reasoned that one or more components of the rich medium might inhibit growth potentially by interfering with the folate pathway. A candidate was yeast extract (YE) that is present in LB and DSM medium at 5 mg/ml and 2 mg/ml respectively and absent in SMM medium. YE has been shown to inhibit thymine mutants of E. coli strains where the active bactericidal ingredient has been identified as adenosine (40). A second possibility was p-aminobenzoic acid (41), a component of YE and directly involved in the folate pathway (FIG. 14) but also bactericidal to E. coli (42).

The inventors grew strains (thy⁺ and thyA thyB) in LB+thymine with varying levels of YE and found that YE concentrations of ≥2 mg/ml inhibited growth of strains carrying two thy (thyA+thyB) insertions (FIG. 17). The inhibitory activity of YE could therefore explain why in LB medium the thyA thyB double insertion strain failed to grow in the presence of thymine (FIG. 5A). Similarly, in DSM where YE is present at a lower concentration reduced growth was observed (FIG. 5C). Finally, the inhibitory action of YE required the need for a modified growth medium (SOC2) lacking YE for preparation of cells for the electroporation step used in the strain constructions (see Methods). Surprisingly, although YE clearly inhibits, the inventors have been unable to attribute this definitively to either adenosine or p-amnobenzoic acid (data not shown).

### b) Gene Transfer

As mentioned earlier DNA-mediated transformation of competent cells could be used to introduce plasmid DNA into B. subtilis cells. However, for introducing DNA into thyA cells in the 2^{nd} step, electroporation using a modified medium lacking YE yielded higher frequencies of integration.

The inventors found that following the 1^{st} genetic cross two colony types, in equal proportion, were apparent on SMM agar supplemented with thymine and trimethoprim (3 μg/ml) (FIG. 18). Type 1 were large opaque colonies (2-3 mm) and Type 2 colonies were translucent, smaller (1 mm) and grew slowly. All colonies could grow at 46° C. in the presence of thymine indicating prima facie a thyA insertion. However, only about one third of type 1 colonies were found using PCR to carry stable thyA insertions while no type 2 colonies carried insertions. The inventors assume that these colonies able to grow on trimethoprim plates must carry some form of compensatory, yet unstable, mutation/s allowing growth in the presence of the antibiotic.

A second important finding was that for the 2^{nd} genetic transfer recombinants could only be selected on SMM minimal media supplemented with thymine, trimethoprim (6 μg/ml) and CAA. If plated directly onto agar lacking CAA small (<1 mm), slow growing colonies would result but after reculture these were found to have lost the thyB insertion as determined by colony PCR. Even in the presence of CAA all colonies were small and only about 20% of those growing on trimethoprim (6 μg/ml) carried a stable thyB insertion. Work in E. coli as well as B. subtilis has shown that disruption of the folate pathway can lead to depletion of key amino acids as well as purines and pyrimidines (43, 44). Trimethoprim-mediated inactivation of DHFR would deplete intracellular levels of THF, MTHF as well as DHF. In turn this would affect the reversible interconversion of serine and glycine with tetrahydrofolic acid (THF), a vital reaction in the synthesis of purines and catalysed by a serine hydroxymethyltransferase (SHMT or GlyA, (45, 46)). MTHF is also utilised in the final step of the biosynthetic pathways of cysteine and methionine (47) and disruption of the pathway by the thyA thyB alleles could introduce a requirement for methionine.

The inventors measured the growth of a thyA thyB insertion strain SH14 in SMM supplemented with CAA, adenine and thymine (FIG. 19). In the presence of trimethoprim (6 μg/ml) growth was optimal reaching a maximal OD₆₀₀ of ˜2 only in SMM containing CAA while in media containing no CAA or carrying only adenine and/or thymine growth was markedly reduced. In the absence of trimethoprim growth of SH14 remained weak compared to the wild type strain PY79 but was i) superior to that in the presence of the antibiotic, ii) growth was restored to normal fitness only in the presence of CAA. Trimethoprim therefore disrupts the folate pathway significantly reducing strain fitness and this could not be restored by supplementation with purines or pyrimidines but only with CAA.

Therefore, for the second genetic transfer step (i.e., for construction of the double, thyA thyB, insertion) selective media may preferably provide all amino acids. However, once constructed the use of trimethoprim is no longer required and strains can be cultivated on any media so long as three criteria are met, first, that the media contains thymine or thymidine, second, YE is either absent or at a concentration less than 2 mg/ml, and third, amino acids are provided in the growth medium.

### c) Choice of One Coat Protein Anchor

For expression of heterologous proteins on the spore surface the coat proteins CotB and CotC can be used for both mono or divalent expression. As expected, when using two different spore coat anchors expression of a protein was higher than using expression from only one. So, as shown in FIG. 4A, TcdA₂₆₋₃₉ levels were higher in SH14 spores carrying thyA::cotB-tcdA₂₆₋₃₉ and thyB::cotC-tcdA₂₆₋₃₉ insertions than in SH13 spores carrying only a thyA::cotB-tcdA₂₆₋₃₉ insertion. Interestingly, using fusion of VP28 to CotB and insertion of this chimera at the thyA locus alone (strain AC01) or at both the thyA and thyB loci (strain AC02) lead to higher levels of expression in the latter. This finding requires some consideration since it must be assumed that each spore would carry a defined number of CotB monomers that could assemble onto the spore surface and simply increasing the number should not lead to higher levels of incorporation in the coat. These strains would however, carry an intact cotB gene (residing at its normal chromosomal locus) so in cells carrying a thyA::cotB-VP28 insertion (i.e., AC01) 50% of displayed CotB proteins should present a wild type CotB and 50% CotB-VP28. In a double thyA thyB insertion strain (AC02) the inventors would predict ˜66% of displayed CotB proteins would present VP28 and ˜33% wild type CotB. This stoichiometry ratio of CotB-VP28 and CotB would agree with the ELISA detection of VP28 as shown in FIG. 4B.

### Example 3

### Reversion

An insertion generated by a double-crossover recombinational event should be inherently stable yet there exists the possibility of acquisition of a compensatory suppressor or bypass mutation. To address this the inventors conducted an experiment to determine whether upon repeated culture in the absence of any selective pressure the thymine dependence could be lost. As shown in Table 2 repeated culture and reculture of SH14 (thyA::cotB-tcdA₂₆₋₃₉ thyA::cotC-tcdA₂₆₋₃₉) yielded no loss of the thymine dependency showing the insertions were stable and suggesting that the acquisition of compensatory mutations, if they were to occur, must be an extremely rare event.

TABLE 2
Reversion¹

| | |
|---|---|
| CFU Culture 1 | CFU Culture 2 |

| | DSM + | | DSM + | |
|---|---|---|---|---|
| Culture | thymine | DSM | thymine | DSM |
| 1st | 2.8 × 10⁸ | 0 | 2.1 × 10⁸ | 0 |
| 2nd | 1.8 × 10⁸ | 0 | 3.4 × 10⁸ | 0 |
| 3rd | 1.85 × 10⁸ | 0 | 1.73 × 10⁸ | 0 |
| 4th | 1.82 × 10⁸ | 0 | 1.6 × 10⁸ | 0 |
| 5th | 1.54 × 10⁸ | 0 | 2.4 × 10⁸ | 0 |

¹Two 25 ml cultures of SH14 (thyA::cotB-tcdA₂₆₋₃₉ thyA::cotC-tcdA₂₆₋₃₉) were made in DSM + thymine (50 μg/ml). The culture was grown for 24 h at 37° C. and a sample analyzed for CFU on either DSM agar with or without thymine (50 μg/ml). A sample was also used to subculture fresh medium (25 ml) and the process repeated four times. CFU/ml for each reculture are shown.

### Example 4

In Vivo Fate of thyA⁻ thyB⁻ Spores in the GI-Tract

It has been demonstrated that for E. coli to colonise the murine GI-tract synthesis of purines and pyrimidines is necessary (48). This implies that the low levels of purines and pyrimidines that might result from digested food or from spurious lysis of resident gut microbiota would not be sufficient to permit growth of a B. subtilis thyA thyB mutant. In humans the intestinal concentration of thymidine is estimated as 0.075 μM and in pigs ˜1.0 μM (49). The inventors gave mice a single oral dose of 2×10⁹ spores of SH250 (thyA⁺ thyB⁺ Cm^{R}) or SH14 (thyA::cotB-tcdA₂₆₋₃₉ thyA::cotC-tcdA₂₆₋₃₉) spores by intra-gastric gavage. Subsequent shedding of heat-resistant spores in freshly voided faeces was determined. For SH250 the strain carried a silent insertion of the cat gene (chloramphenicol acetyltransferase) so after serial dilution heat resistant CFU was determined on chloramphenicol plates. SH14 spores were shed in the faeces and could only be detected on agar plates supplemented with thymine but when plated on plates lacking thymine no CFU could be detected (FIG. 6). After 10 days the number of SH14 spores being shed in the faeces was at levels (<10³/g of faeces) at the threshold of detection. This in vivo data shows that the kinetics of spore shedding of thyA⁻ thyB⁻ spores are indistinguishable from shedding of spores of a wild type strain. The inventors have confirmed that SH14 spores germinate efficiently and are equivalent to wild type spores (data not shown). Although it is not possible to definitively determine whether SH14 spores could proliferate in the GI-tract it was possible to show that thyA thyB spores that transited the GI-tract were unable to survive in the absence of thymine. Also, there was no evidence of reversion or acquisition of markers that might enable germinated spores to survive.

### Example 5

### Utility of Spore Display

The inventors used three examplars to demonstrate that spores carrying insertions at the thyA and thyB loci were suitable for applied purposes, as vaccine delivery vehicles, for conjugation of proteins to the spore surface and finally for display of active enzymes. As vaccines the inventors used SH14 spores that express the TcdA₂₆₋₃₉ antigen of C. difficile fused to two spore coat protein anchors, CotB and CotC. SH14 is equivalent to strain PP108 that has previously been shown to confer protection to C. difficile infection (CDI) in murine and hamster models of infection (4, 38). The inventors immunised mice with spores of SH14 and PP108 using oral administration and after a total of four doses measured TcdA₂₆₋₃₉-specific IgG (FIG. 7A) and IgA (FIG. 7B) in the serum and faeces respectively. Compared to control groups (naive and mice dosed with naked PY79 spores) that exhibited no responses, both PP108 and SH14 spores generated high titres of IgG and IgA and based on previous work the inventors would predict these levels of antibodies would be protective.

For conjugation of proteins to streptavidin displayed on the spore surface the inventors first biotinylated a rabbit polyclonal TdA₂₆₋₃₉-specific antibody and demonstrated that the antibody conjugated to SH16 spores using immunofluorescence detection (FIG. 8A) and also confirmed using whole spore ELISA (not shown). Using these conjugated spores the inventors asked whether SH16 spores displaying TcdA₂₆₋₃₉ IgG could subtract C. difficile toxins from a crude cell-free lysate. As shown in Table 3 incubation of conjugated spores with toxin-containing lysates for just 5 min. reduced toxicity by 90%. Interestingly, PY79 spores also had some ability to bind TdA₂₆₋₃₉ antibodies and were able to provide a modest reduction (10-20%) in toxin activity. Used as an example this experiment demonstrates that spores might have potential for therapeutic purposes, for example in the oral administration of antibodies.

TABLE 3
Subtraction of C. difficile toxins¹

| Mixture | Cytotoxicity (%) |
|---|---|
| Medium only | 0 |
| Toxins only | 100 |
| PY79 treated spores + toxins | 80-90 |
| SH16 conjugated spores + toxin | 10 |

¹Cytoxicity in HT29 cells was measured using crude toxins from C. difficile or from the same toxins pretreated with SH16 spores conjugated with a polyclonal antibody to TcdA₂₆₋₃₉ or PY79 treated spores.

Enzymes are commonly included in animal feeds where they improve digestion and nutrition. Proteases and amylases are pertinent examples used here to show that it is possible to display an enzyme that retains activity on the spore surface. SH18 spores expressing amylase were found able to express active amylase on their surface (FIG. 8B). Using casein agar we showed that SH20 spores expressing the alkaline protease, subtilisin E, carried enzymatic activity (FIG. 8B). In liquid suspensions we found that 10¹⁰ spores of SH20 had ˜0.127 units of protease activity.

### Summary of Examples 1 to 5

The inventors have described a straightforward method to contain genetically modified bacterial spores. Their approach expands yet differs from those described for Lactobacillus acidophilus (51) and Lactococcus lactis (Steidler et al., 2003) that rely on the indigenous suicide resulting from a ‘thymine-less death’. First described in 1954 (23) thymine dependence differs from other auxotrophies in that the absence of thymine is bactericidal and so bacteria carrying defects in the thymidylate synthase genes cannot accumulate in the environment. Bacillus species carry two thymidylate synthase genes (thyA and thyB) requiring inactivation of both loci to achieve complete dependence on thymine. The inventors have shown here that this is possible and have identified a two-step cloning procedure requiring insertional inactivation of first thyA and then thyB loci. Their approach does not require the introduction of antibiotic resistance markers for selection but rather the development of increasing levels of resistance to trimethoprim that arise from successive disruption of the folate pathway. Coupled with the temperature sensitive phenotype of insertions at the thyA and thyB loci this provides a useful method for both selection and screening of insertion although technically there are a number of constraints that must be considered. The inventors show that the absence of thymine is bactericidal and observe no evidence for reversion or suppression despite repeated passage of these strains. Of course, the purpose of the cloning system is to construct strains able to express proteins for applied purposes, for example, for expression of heterologous antigens or enzymes. The inventors have used examples here showing that chimeric proteins comprised of a foreign protein fused to a spore coat protein can be displayed on the spore surface. This has included the delivery of two enzymes (subtilisin E and α-amylase), putative vaccine protective antigens as well as streptavidin which was used to conjugate to a polyclonal antibody. It is clear though that this system could equally be used for expression of proteins in, or secretion from, the vegetative cell.

### REFERENCES

1. Nicholson W J, Munakata N, Horneck G, Melosh H J, Setlow P. 2000. Resistance of Bacillus endospores to extreme terrestial and extraterrestrial environments. Microbiology and Molecular Biology Reviews 64:548-572.
2. Pan J G, Kim E J, Yun C H. 2012. Bacillus spore display. Trends Biotechnol 30:610-612.
3. Duc L H, Hong H A, Fairweather N, Ricca E, Cutting S M. 2003. Bacterial spores as vaccine vehicles. Infection and Immunity 71:2810-2818.
4. Permpoonpattana P, Hong H A, Phetcharaburanin J, Huang J M, Cook J, Fairweather N F, Cutting S M. 2011. Immunization with Bacillus spores expressing toxin A peptide repeats protects against infection with Clostridium difficile strains producing toxins A and B. Infection and immunity 79:2295-2302.
5. Lee S, Belitsky B R, Brinker J P, Kerstein K O, Brown D W, Clements J D, Keusch G T, Tzipori S, Sonenshein A L, Herrmann J E. 2010. Development of a Bacillus subtilis-based rotavirus vaccine. Clin Vaccine Immunol 17:1647-1655.
6. Fu L L, Li W F, Du H H, Dai W, Xu Z R. 2008. Oral vaccination with envelope protein VP28 against white spot syndrome virus in Procambarus clarkii using Bacillus subtilis as delivery vehicles. Lett Appl Microbiol 46:581-586.
7. Nguyen A T, Pham C K, Pham H T, Pham H L, Nguyen A H, Dang L T, Huynh H A, Cutting S M, Phan T N. 2014. Bacillus subtilis spores expressing the VP28 antigen: a potential oral treatment to protect Litopenaeus vannamei against white spot syndrome. FEMS Microbiol Lett 358:202-208.
8. Ning D, Leng X, Li Q, Xu W. 2011. Surface-displayed VP28 on Bacillus subtilis spores induce protection against white spot syndrome virus in crayfish by oral administration. Journal of applied microbiology 111:1327-1336.
9. Valdez A, Yepiz-Plascencia G, Ricca E, Olmos J. 2014. First Litopenaeus vannamei WSSV 100% oral vaccination protection using CotC::Vp26 fusion protein displayed on Bacillus subtilis spores surface. J Appl Microbiol doi:10.1111/jam.12550.
10. EFSA. 2008. The maintenance of the list of QPS microorganisms intentionally added to food or feed. EFSA Journal 923:1-48.
11. Hong H A, Duc le H, Cutting S M. 2005. The use of bacterial spore formers as probiotics. FEMS Microbiol Rev 29:813-835.
12. Amalaradjou M A, Bhunia A K. 2013. Bioengineered probiotics, a strategic approach to control enteric infections. Bioengineered 4:379-387.
13. Sorokulova I B, Beliavskaia V A, Masycheva V A, Smirnov V V. 1997. Recombinant probiotics: problems and prospects of their use for medicine and veterinary practice. Vestn Ross Akad Med Nauk:46-49.
14. Potot S, Serra C R, Henriques A O, Schyns G. 2010. Display of recombinant proteins on Bacillus subtilis spores, using a coat-associated enzyme as the carrier. Appl Environ Microbiol 76:5926-5933.
15. Nguyen V A, Huynh H A, Hoang T V, Ninh N T, Pham A T, Nguyen H A, Phan T N, Cutting S M. 2013. Killed Bacillus subtilis spores expressing streptavidin: a novel carrier of drugs to target cancer cells. J Drug Target 21:528-541.
16. Shimotsu H, Henner D J. 1986. Construction of a single copy integration vector and its use in analysis of regulation of the trp operon of Bacillus subtilis. Gene 43:85-94.
17. Guerout-Fleury A M, Frandsen N, Stragier P. 1996. Plasmids for ectopic integration in Bacillus subtilis. Gene 180:57-61.
18. Bloor A E, Cranenburgh R M. 2006. An efficient method of selectable marker gene excision by Xer recombination for gene replacement in bacterial chromosomes. Appl Environ Microbiol 72:2520-2525.
19. Iwanicki A, Piatek I, Stasilojc M, Grela A, Lega T, Obuchowski M, Hine K. 2014. A system of vectors for Bacillus subtilis spore surface display. Microb Cell Fact 13:30.
20. Cano R J, Borucki M. 1995. Revival and identification of bacterial spores in 25- to 40-million-year-old Dominican amber. Science 268:1060-1064.
21. Nicholson W L. 2002. Roles of Bacillus endospores in the environment. Cellular and Molecular Life Sciences 59:410-416.
22. Mauriello E M, Cangiano G, Maurano F, Saggese V, De Felice M, Rossi M, Ricca E. 2007. Germination-independent induction of cellular immune response by Bacillus subtilis spores displaying the C fragment of the tetanus toxin. Vaccine 25:788-793.
23. Cohen S S, Barner H D. 1954. Studies on Unbalanced Growth in Escherichia coli. Proc Natl Acad Sci USA 40:885-893.
24. Goulian M, Bleile B M, Dickey L M, Grafstrom R H, Ingraham H A, Neynaber S A, Peterson M S, Tseng B Y. 1986. Mechanism of thymineless death. Adv Exp Med Biol 195 Pt B:89-95.
25. Ahmad S I, Kirk S H, Eisenstark A. 1998. Thymine metabolism and thymineless death in prokaryotes and eukaryotes. Annu Rev Microbiol 52:591-625.
26. Rolfe R. 1967. On the mechanism of thymineless death in Bacillus subtilis. Proc Natl Acad Sci USA 57:114-121.
27. Neuhard J, Price A R, Schack L, Thomassen E. 1978. Two thymidylate synthetases in Bacillus subtilis. Proc Natl Acad Sci USA 75:1194-1198.
28. Zeigler D R, Pragai Z, Rodriguez S, Chevreux B, Muffler A, Albert T, Bai R, Wyss M, Perkins J B. 2008. The Origins of 168, W23, and Other Bacillus subtilis Legacy Strains. Journal of Bacteriology 190:6983-6995.
29. Harwood C R, Cutting S M. 1990. Molecular Biological Methods for Bacillus. John Wiley & Sons Ltd., Chichester, England.
30. Nicholson W L, Setlow P. 1990. Sporulation, germination and outgrowth., p 391-450. In Harwood. C R, Cutting. S M (ed), Molecular Biological Methods for Bacillus. John Wiley & Sons Ltd., Chichester, U K.
31. Ikemura H, Takagi H, Inouye M. 1987. Requirement of pro-sequence for the production of active subtilisin E in Escherichia coli. J Biol Chem 262:7859-7864.
32. Tran T I, Mamo G, Mattiasson B, Hatti-Kaul R. 2010. A thermostable phytase from Bacillus sp. MD2: cloning, expression and high-level production in Escherichia coli. J Ind Microbiol Biotechnol 37:279-287.
33. Dubnau D, Davidoff-Abelson R. 1971. Fate of transforming DNA following uptake by competent Bacillus subtilis. I. Formation and properties of the donor-recipient complex. Journal of Molecular Biology 56:209-221.
34. Cutting S M, Vander-Horn P B. 1990. Genetic Analysis, p 27-74. In Harwood C R, Cutting S M (ed), Molecular Biological Methods for Bacillus. John Wiley & Sons Ltd., Chichester, England.
35. Sun Q Y, Ding L W, He L L, Sun Y B, Shao J L, Luo M, Xu Z F. 2009. Culture of Escherichia coli in SOC medium improves the cloning efficiency of toxic protein genes. Anal Biochem 394:144-146.
36. Permpoonpattana P, Phetcharaburanin J, Mikelsone A, Dembek M, Tan S, Brisson M C, La Ragione R, Brisson A R, Fairweather N, Hong H A, Cutting S M. 2013. Functional Characterization of Clostridium difficile Spore Coat Proteins. J Bacteriol 195:1492-1503.
37. Harwood C R, Coxon R D, Hancock I C. 1990. The Bacillus cell envelope and secretion, p 327-390. In Harwood C R, Cutting S M (ed), Molecular Biological Methods for Bacillus. John Wiley & Sons, Chichester.
38. Hong H A, Hitri K, Hosseini S, Kotowicz N, Bryan D, Mawas F, Wilkinson A J, van Broekhoven A, Kearsey J, Cutting S M. 2017. Mucosal Antibodies to the C-terminus of Toxin A Prevent Colonization of Clostridium difficile Infection and Immunity 85.
39. Walsh G A, Power R F, Headon D R. 1993. Enzymes in the animal-feed industry. Trends Biotechnol 11:424-430.
40. Kinoshita N, Akiyoshi H, Endo H. 1969. Isolation and characterization of substance in yeast extract which inhibits growth of thymine-less strains of Escherichia coli. J Gen Microbiol 59:393-400.
41. Blanchard K C. 1941. The isolation of p-aminobenzoic acid from yeast. The Journal of Biological Chemistry 140:919-926.
42. Davis B D. 1951. Inhibition of Escherichia coli by p-aminobenzoic acid and its reversal by p-hydroxybenzoic acid. J Exp Med 94:243-254.
43. Amyes S G, Smith J T. 1974. Trimethoprim action and its analogy with thymine starvation. Antimicrob Agents Chemother 5:169-178.
44. Stepanek J J, Schakermann S, Wenzel M, Prochnow P, Bandow J E. 2016. Purine biosynthesis is the bottleneck in trimethoprim-treated Bacillus subtilis. Proteomics Clin Appl 10:1036-1048.
45. Schirch L. 1982. Serine hydroxymethyltransferase. Adv Enzymol Relat Areas Mol Biol 53:83-112.
46. Ponce-de-Leon M M, Pizer L I. 1972. Serine biosynthesis and its regulation in Bacillus subtilis. J Bacteriol 110:895-904.
47. Ferla M P, Patrick W M. 2014. Bacterial methionine biosynthesis. Microbiology 16:1571-1584.
48. Vogel-Scheel J, Alpert C, Engst W, Loh G, Blaut M. 2010. Requirement of purine and pyrimidine synthesis for colonization of the mouse intestine by Escherichia coli. Appl Environ Microbiol 76:5181-5187.
49. Steidler L, Neirynck S, Huyghebaert N, Snoeck V, Vermeire A, Goddeeris B, Cox E, Remon J P, Remaut E. 2003. Biological containment of genetically modified Lactococcus lactis for intestinal delivery of human interleukin 10. Nat Biotechnol 21:785-789.
50. Yan S, Wu G. 2017. Bottleneck in secretion of alpha-amylase in Bacillus subtilis. Microb Cell Fact 16:124.
51. Fu X, Xu J G. 2000. Development of a chromosome-plasmid balanced lethal system for Lactobacillus acidophilus with thyA gene as selective marker. Microbiol Immunol 44:551-556.
52. CLSI. 2010. Methods for antimicrobial dilution and disk susceptibility testing of infrequently isolated or fastidious bacteria; approved guideline—Second Ed M45-A2. Institute CaLS, USA.
53. Barra-Carrasco, J., V. Olguin-Araneda, A. Plaza-Garrido, C. Miranda-Cardenas, G. Cofre-Araneda, M. Pizarro-Guajardo, M. R. Sarker & D. Paredes-Sabja, (2013) The Clostridium difficile exosporium cysteine (CdeC)-rich protein is required for exosporium morphogenesis and coat assembly. J Bacteriol 195: 3863-3875.
54. Permpoonpattana, P., E. H. Tolls, R. Nadem, S. Tan, A. Brisson & S. M. Cutting, (2011b) Surface layers of Clostridium difficile endospores. J Bacteriol 193: 6461-6470.
55. Phetcharaburanin, J., H. A. Hong, C. Colenutt, I. Bianconi, L. Sempere, P. Permpoonpattana, K. Smith, M. Dembek, S. Tan, M. C. Brisson, A. R. Brisson, N. F. Fairweather & S. M. Cutting, (2014) The spore-associated protein BclA1 affects the susceptibility of animals to colonization and infection by Clostridium difficile. Mol Microbiol 92: 1025-1038.

1. A process for introducing at least one heterologous gene into a spore-forming bacterium and rendering the bacterium unable to proliferate in the absence of thymine or thymidine, the process comprising:
(i) introducing a heterologous gene into a thymidylate synthase gene within a spore-forming bacterium; and
(ii) growing said spore-forming bacterium in the presence of trimethoprim.

2. The process of claim 1, wherein the spore-forming bacterium is (i) Bacillus Spp., preferably B. subtilis, or (ii) Clostridium Spp., preferably C. difficile.

3. The process claim 1, wherein:
step (i) is the introduction of a heterologous gene into the thyA gene and the thyB gene in a Bacillus Spp.

4. The process of claim 1, wherein:
step i) is the introduction of a heterologous gene into either the thyA gene or the thyB gene in a Bacillus Spp. and preferably further comprising:
(iii) introducing a heterologous gene into the other of the thyA gene or the thyB gene in said Bacillus Spp.; and
(iv) growing said Bacillus Spp. in the presence of trimethoprim.

5. (canceled)

6. The process of claim 4, wherein:
step (i) is the introduction of a heterologous gene into the thyA gene in a Bacillus Spp.; and
step (iii) is the introduction of a heterologous gene into the thyB gene in a Bacillus Spp.

7. The process of claim 1, wherein:
during step (ii), the spore-forming bacterium is grown in a medium comprising yeast extract (YE) at a concentration lower than 2 mg/ml, 1.5 mg/ml, 1 mg/ml, 0.5 mg/ml, 0.25 mg/ml, 0.125 mg/ml, 0.1 mg/ml, 0.01 mg/ml, 0.001 mg/ml, 0.0001 mg/ml, or 0 mg/ml, preferably the medium in which the spore-forming bacterium is grown contains no YE.

8. The process of claim 4, wherein:
during step (iv), the Bacillus Spp. is grown in a medium comprising yeast extract (YE) at a concentration lower than 2 mg/ml, 1.5 mg/ml, 1 mg/ml, 0.5 mg/ml, 0.25 mg/ml, 0.125 mg/ml, 0.1 mg/ml, 0.01 mg/ml, 0.001 mg/ml, 0.0001 mg/ml, or 0 mg/ml, preferably the medium in which the Bacillus Spp. is grown contains no YE.

9. The process of claim 1, wherein:
during step (ii), the spore-forming bacterium is grown in a medium comprising thymine or thymidine.

10. The process of claim 4, wherein:
during step (iv), the Bacillus Spp. is grown in a medium comprising thymine or thymidine.

11. The process of claim 4, wherein:
the amount of trimethoprim present in step (iv) is higher than the amount of trimethoprim present in step (ii).

12. The process of claim 5, wherein cells carrying an insertion at the thyA locus are verified by the ability to grow at 37° C. with or without thymine or thymidine but the inability to grow at 46° C. unless supplemented with thymine or thymidine.

13. The process of claim 1, wherein:
YE is present at a concentration lower than 2 mg/ml, or preferably not present, in the media used at any stage of the process.

14. The process of claim 4, wherein:
during step (iv), the Bacillus Spp. is grown in a medium supplemented with at least one amino acid, preferably wherein:
during step (iv), the Bacillus Spp. is grown in a medium comprising casamino acids (CAA).

15. (canceled)

16. The process of claim 2, wherein:
cells carrying two insertions are verified by the inability to grow at 37° C. or 46° C. unless supplemented with thymine or thymidine.

17. (canceled)

18. The process of claim 1, further comprising:
forming a bacterial spore from said spore-forming bacterium.

19. A bacterial spore obtained or obtainable by the process of claim 1, preferably wherein the bacterial spore is Bacillus spp. and comprises non-functional thyA and thyB genes, preferably due to the introduction of heterologous genes therein.

20. (canceled)

21. A bacterial spore comprising at least one heterologous gene and unable to proliferate in the absence of thymine or thymidine, wherein the bacterial spore is produced by a process comprising:
(i) introducing a heterologous gene into a thymidylate synthase gene within a spore-forming bacterium;
(ii) growing said spore-forming bacterium in the presence of trimethoprim; and
(iii) forming a bacterial spore from said spore-forming bacterium.

22. (canceled)

23. (canceled)

24. (canceled)

25. (canceled)
